(19) United States
(12) Patent Application Publication
(10) Pub. No.: US 2024/0148809 A1
(43) Pub. Date: May 9, 2024

(54) CANNABIS PREVENTS NK INACTIVATION IN CANCER AND INCREASES NK FUNCTION

(71) Applicants: The Regents of the University of California, Oakland, CA (US); Tracy Ryan, Sherman Oaks, CA (US)

(72) Inventors: Anahid Jewett, Los Angeles, CA (US); Tracy Ryan, Sherman Oaks, CA (US)

(21) Appl. No.: 18/025,499

(22) PCT Filed: Sep. 9, 2021
(86) PCT No.: PCT/US21/49599

§ 371 Date: Mar. 9, 2023

Related U.S. Application Data
(60) Provisional application No. 63/076,126, filed on Sep. 9, 2020.

(51) Int. Cl. A61K 36/185; A61K 45/06; A61P 35/00; A61P 37/04; C07K 14/54; C07K 16/28

(52) U.S. Cl. CPC A61K 36/185; A61K 45/06; A61P 35/00; A61P 37/04; C07K 14/54; C07K 16/2809; C07K 16/2818; C07K 16/283; A61K 2039/507

(57) ABSTRACT

The present invention provides methods for increasing NK cytotoxicity of natural killer (NK) cells by administering to a subject in need thereof a composition comprising cannabis in an amount effective to induce apoptosis of a tumor cell. This invention also provides methods for increasing or restoring secretion of interferon gamma (IFN-γ) by a peripheral blood mononuclear cell (PMBC), in particular an NK cell, by administering to a subject in need thereof a composition comprising cannabis. The present invention further provides methods for preventing or reversing inactivation of cytotoxicity of an NK cell by administering to a subject in need thereof a composition comprising cannabis in an amount effective to induce apoptosis of a tumor cell. Methods for treating a subject having cancer or a tumor by administering a composition comprising cannabis are provided.

### CROSS-REFERENCE TO RELATED APPLICATIONS

This application claims the benefit of U.S. Provisional Application No. 63/076,126, filed on Sep. 9, 2020, the entire contents of which are incorporated herein in their entirety by this reference.

### BACKGROUND OF THE INVENTION

The innate immune system includes white blood cells, leukocytes, including phagocytes, macrophages, mast cells, neutrophils, eosinophils, basophils and natural killer cells (“NK cells”) and dendritic cells. NK cells belong to cytotoxic lymphocytes expressing CD56 and CD16 surface proteins, capable of killing cancer and virus-infected cells by spontaneous cytolytic activity without any priming (prior immunization) or prior activation, unlike cytotoxic T cells, which require priming by antigen presenting cells. NK cells detect the presence of compromised cells, i.e., physiologically stressed or abnormal cells, such as malignant (neoplastic) cells and virus-infected cells, by monitoring the level of class I MHC (also called “MHC I”) glycoproteins, expressed on the surface of almost all nucleated cells. The presence of high levels of these proteins inhibits the killing activity of NK cells; normal healthy cells express MHC I receptors which mark these cells as “self”. Inhibitory receptors on the surface of the NK cell recognize the MHC I receptors, “switch off” the NK cells, and thus, prevent them from killing healthy cells. NK cells selectively kill target cells expressing abnormally low MHC I levels (downregulated expression of self MHC I) and thus recognized as “missing self”, including both virally-infected cells and some cancer cells. NK cells can use two distinct mechanisms to kill their target cells, either by cytotoxic granule exocytosis or by induction of death receptor-mediated apoptosis. When the former mechanism occurs, NK cells release cytotoxic granules containing perforin and granzymes, which leads to lysis of the target cells.

Furthermore, NK cells secrete inflammatory cytokines, primarily interferon gamma (IFN-γ) and Tumour Necrosis Factor alpha (TNF alpha), and induce target cell cytolysis. It has been shown that IFN-γ and TNF-α synergistically enhance NK cell cytotoxicity through NF-κB-dependent up-regulation of ICAM-1 expression in target cells, thus promoting their conjugate formation with NK cells. IFNγ is a key cytokine for innate and adaptive immunity; IFNγ activates macrophages and induces Class II major histocompatibility complex (MHC) molecule expression. IFNγ also regulates various aspects of immune system responses, including NK cell actions, by forming a positive feedback loop.

Evidence suggests that the cytotoxic function of immune effectors is largely suppressed in the tumor microenvironment. NK cells undergo exhaustion (functional allergy) after performing their killing of target cells. Studies have shown that after an exposure to target cells, NK cells go through inactivation, loose their cytotoxic function and become apoptotic. In addition to NK cytotoxic function becoming inactivated in cancer patients, NK cells demonstrate decreased secretion of cytokines, in particular IFNγ.

Therefore, a critical need exists for improving NK cell antitumor function, for utilizing enhanced NK tumor cell killing to develop therapeutically effective compositions and methods for treating cancer in a subject, in particular solid tumors.

### SUMMARY OF THE INVENTION

The present disclosure is based at least in part on the discovery that cannabis is effective in activating NK cells, e.g., increasing the NK cells' secretion of a cytokine (e.g., IFN-γ) or increasing the cytotoxicity of NK cells.

In certain aspects, provided herein is A method for increasing cytotoxicity of a natural killer (NK) cell, the method comprising administering to a subject a composition comprising cannabis.

In certain aspects, also provided herein is a method for increasing or restoring secretion of interferon gamma (IFN-γ) by a peripheral blood mononuclear cell (PMBC), the method comprising administering to a subject a composition comprising cannabis. In some embodiments, the PMBC is a natural killer (NK) cell, a natural killer T (NKT), a CD4 T helper (TH) lymphocyte, and/or a CD8 cytotoxic cell. In some embodiments, the PMBC is a natural killer (NK) cell. In some embodiments, the composition comprising cannabis is administered in an amount effective to reactivate an exhausted NK cell.

In certain aspects, provided herein is a method for preventing or reversing inactivation of cytotoxicity of a natural killer (NK) cell (e.g., an exhausted NK cell), the method comprising administering to a subject a composition comprising cannabis.

In certain aspects, further provided herein is a method for treating a subject having cancer and/or a tumor, the method comprising: (a) obtaining a blood or tissue sample of the subject; (b) determining a level of activation of an NK cell and/or a level of secretion of IFN-γ by a NK cell in the blood or tissue sample of the subject; (c) comparing the determined level of activation of the NK cell and/or the determined level of secretion of IFN-γ by the NK cell to a control, optionally wherein the control is a level of activation of an NK cell from a healthy subject and/or a level of secretion of IFN-γ by a NK cell in the blood or tissue sample of a healthy subject; wherein when the determined level of activation of the NK cell and/or the determined level of secretion of IFN-γ by the NK cell in the blood or tissue sample of the subject is decreased compared to the control, the method further comprises administering to the subject a composition comprising cannabis.

In certain aspects, provided herein is a method of increasing cytotoxicity of an NK cell or a method of increasing secretion of IFN-γ by the NK cell, the method comprising contacting the NK cell with cannabis. In some embodiments, the NK cell is contacted with cannabis in vivo, in vitro, or ex vivo.

Numerous embodiments are further provided that can be applied to any aspect of the present invention and/or combined with any other embodiment described herein.

For example, in some embodiments, the method of the present disclosure further comprises administering to the subject: (a) a composition comprising at least one of anti-CD3 antibodies, anti-CD28 antibodies, IL-2, IL-2 and anti-CD16 antibodies, and IL-2 and sAJ2 bacteria, or a combination thereof, optionally wherein the antibodies are monoclonal antibodies; and/or (b) at least one cancer therapy.

In some embodiments, the composition comprising cannabis is administered by inhalation, oral administration, sublingual administration, and/or topical administration.

In some embodiments, the oral administration of the composition comprising cannabis comprises ingesting (a) a cannabis oil; (b) a cannabis tincture; and/or (c) a food and/or beverage, wherein the food and the beverage each comprises the composition comprising cannabis.

In some embodiments, the inhalation is by smoking or vaporizing.

In some embodiments, the composition comprising at least one of anti-CD3 antibodies, anti-CD28 antibodies, IL-2, IL-2 and anti-CD16 antibodies, and IL-2 and sAJ2 bacteria is administered intravenously.

In some embodiments, the composition comprising cannabis and the composition comprising at least one of anti-CD3 antibodies, anti-CD28 antibodies, IL-2, IL-2 and anti-CD16 antibodies, and IL-2 and sAJ2 bacteria are administered concurrently or sequentially.

In some embodiments, the cannabis comprises CBD, CBDa, THC and/or THCa.

In some embodiments, the method comprises administering to the subject at least about 0.001 mg/kg and no more than about 100 mg/kg of cannabis.

In some embodiments, the subject is administered cannabis at least once a week or at least once a day.

In some embodiments, the subject has cancer and/or a tumor.

In some embodiments, the subject demonstrates decreased secretion of IFN-γ prior to the administration of the composition comprising cannabis.

Other features and advantages of the present invention will become apparent from the following detailed description, examples and figures. It should be understood, however, that the detailed description and the specific examples while indicating certain embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1A-FIG. 1B show the treatment of oral squamous carcinoma cells (OSCCs) with different concentrations of Win55,2122-2. FIG. 1A and Table 1 show the levels of decrease in Forward Scatter (FS) in the cells representing loss of cell morphology closely associated with the induction of cell death. FIG. 1B and Table 1 show the determination of cell death using Propidium Iodide (PI) staining. As can be seen in FIG. 1A and Table 1 Win55,2122-2 decreased FS even at the lower concentrations of drug, and a plateauing effect was seen until 5 μM levels. When cell death was determined by staining with PI significant cell death was also observed at higher concentrations and titration could be seen at 25 and 5 μM of Win55,2122-2. Therefore, Win55,2122-2 is a potent inducer of cell death in differentiated oral tumors.

FIG. 2 shows the treatment of stem like oral squamous carcinoma cells (OSCSCs) with different concentrations of Win55,2122-2 and the levels of decrease in Forward Scatter (FS) in the cells, as well as determination of cell death using Propidium Iodide (PI) staining. As can be seen in the figure Win55,2122-2 did not change FS even at the higher concentrations of drug. When cell death was determined by staining with PI no or moderate levels of cell death was observed even at higher concentrations of Win55,2122-2 when compared to untreated or DMSO treated tumors. Therefore, Win55,2122-2 does not induce significant cell death in OSCSCs unlike OSCCs.

FIG. 3 shows the results of flow cytometry that was performed to determine the proportion of NK, CD3, CD4, CD8, CD19, and CD14 cells, as well as the ratio of CD4/CD8 in PBMC. Percent NK cells and CD4/CD8 ratio in patients' (“P”) PBMC is significantly higher than healthy (“H”) donors, whereas patient PBMC contains lower percentage of total CD3+ T cells as well as lower CD8+ T cell than healthy donors.

FIG. 4A-FIG. 4C show increased NK cell cytotoxicity from patients on Cannabis as compared to healthy controls when compared to data from patients without the use of Cannabis. PBMCs (1×10⁶ cells/ml) from healthy donors and from cancer patients with and without Cannabis use were left untreated (“unt”) or treated with IL-2 (1000 U/ml), or the combination of IL-2 and anti-CD16mAb (3 μg/ml) or IL-2 and combination of anti-CD3/CD28 antibodies, and IL-2 in combination with sAJ2 (20:1, bacteria:PBMCs) for 18 hours before they were added to 51 Cr labeled oral squamous cell carcinoma stem cells (OSCSCs) at various effector to target ratios. NK cell-mediated cytotoxicity using a standard 4-hour 51 Cr release assay against the oral squamous cell carcinoma stem cell line (OSCSCs) were performed and the lytic units 30/10⁶ cells were determined using the inverse number of lymphocytes required to lyse 30% of OSCSCs×100. FIGS. 4A-4C show results of the chromium release assay that was performed with PBMC obtained from Cannabis Patients (FIG. 4A), All Cancer Patients (FIG. 4B), and Pancreatic Cancer Patients (FIG. 4C).

FIG. 5A-FIG. 5C show increased IFN-γ spots in PBMCs from patients on Cannabis as compared to healthy controls when compared to data from patients without the use of Cannabis. PBMCs were isolated as described in the Materials & Methods (M&M) section and Elispot was conducted to determine the numbers of IFN-γ spots representing the number of cells expressing IFN-γ. PBMCs were treated with IL-2 (1000 U/ml) or the combination of IL-2 and anti-CD16mAb (3 □g/ml) or IL-2 and combination of anti-CD3/CD28 antibodies or IL-2 in combination with sAJ2. The number of IFN-γ spots was higher in patient's PBMCs on Cannabis when compared to healthy controls. Specifically, treatment with IL-2 and anti-CD16mAb, IL-2+anti-CD3/CD28 antibodies and IL-2+sAJ2. When compared to the sets of patients and healthy without the use of Cannabis the profiles were reversed, with healthy having higher increase in the spots than patients. ELISPOT was performed on PBMCs from Cannabis Patients (FIG. 5A), All Cancer Patients (FIG. 5B), Pancreatic Cancer Patients (FIG. 5C).

FIG. 6A-FIG. 6C show increased IFN-γ release from PBMCs of patients on Cannabis as compared to healthy controls when compared to data from patients without the use of Cannabis. PBMCs were isolated as described in the M&M section and ELISA was conducted to determine the amount of IFN-γ secretion in the supernatants. PBMCs were treated with IL-2 (1000 U/ml) or the combination of IL-2 and anti-CD16mAb (3 μg/ml) or IL-2 and combination of anti-CD3/CD28 antibodies or IL-2 in combination with sAJ2. The levels of IFN-γ secretion were higher in patient's PBMCs on Cannabis when compared to healthy controls. Specifically, treatment with IL-2 and anti-CD16mAb, IL-2+anti-CD3/CD28 antibodies and IL-2 with sAJ2. When compared to the sets of patients and healthy without the use of Cannabis the profiles were reversed, with healthy having higher increase in the spots than patients. ELISA was performed with supernatant from PBMC Cannabis Patients (FIG. 6A), All Cancer Patients (FIG. 6B), Pancreatic Cancer Patients (FIG. 6C).

FIG. 7A-FIG. 7B show increased NK cell cytotoxicity from patients on Cannabis as compared to healthy controls when compared to data from patients without the use of Cannabis. Purified NK cells (1×10⁶ cells/ml) from healthy donors and cancer patients with and without Cannabis use were left untreated or treated with IL-2 (1000 U/ml), or the combination of IL-2 and anti-CD16mAb (3 μg/ml) or IL-2 in combination with sAJ2 (20:1 bacteria: PBMCs) for 18 hours before they were added to 51 Cr labeled oral squamous cell carcinoma stem cells (OSCSCs) at various effector to target ratios. NK cell-mediated cytotoxicity using a standard 4-hour 51 Cr release assay against the oral squamous cell carcinoma stem cell line (OSCSCs) were performed and the lytic units 30/10⁶ cells were determined using the inverse number of lymphocytes required to lyse 30% of OSCSCs×100. Chromium release assay was performed with purified NK cells obtained from Cannabis Patients (FIG. 7A), All Cancer Patients (FIG. 7B).

FIG. 8A-FIG. 8B show increased IFN-γ spots from the NK cells from patients on Cannabis as compared to healthy controls when compared to data from patients without the use of Cannabis. Purified NK cells were isolated as described in the M&M section and Elispot was conducted to determine the numbers of IFN-γ spots representing the number of cells expressing IFN-γ. NK cells were treated with IL-2 (1000 U/ml) or the combination of IL-2 and anti-CD16mAb (3 μg/ml) or IL-2 in combination with sAJ2. The number of IFN-γ spots was variable in patient's NKs on Cannabis when compared to healthy controls. ELISPOT was performed on purified NK cells from Cannabis Patients (FIG. 8A), All Cancer Patients (FIG. 8B).

FIG. 9A-FIG. 9B show increased IFN-γ release from the NK cells from patients on Cannabis as compared to healthy controls when compared to data from patients without the use of Cannabis purified NK cells were isolated as described in the M&M section and ELISA was conducted to determine the amount of IFN-γ secretion in the supernatants. NKs were treated with IL-2 (1000 U/ml) or the combination of IL-2 and anti-CD16mAb (3 μg/ml) or IL-2 in combination with sAJ2. The levels of IFN-γ secretion was variable in patient's NKs on Cannabis when compared to healthy controls. ELISA was performed with purified NK cells of Cannabis Patients (FIG. 9A), All Cancer Patients (FIG. 9B).

FIG. 10A-FIG. 10B show results of an enzyme-linked immunospot (ELISPOT) assay that was conducted to determine the numbers of IFN-γ spots representing the number of cells expressing IFN-γ. Treated (IL2, IL2+antiCD16, IL2+antiCD3/28) and untreated PMBC cells of a breast cancer patient P38 (who was treated with CBD) compared to two healthy control subjects, H43 and H44 (FIG. 10A). Treated (IL2, IL2+antiCD16, IL2+antiCD3/28) and untreated PMBC cells of a breast cancer patient P77 VB (who was treated with CBD) compared to a healthy control subject, H74 (FIG. 10B). Comparable PBMC expression of IFN-γ was shown for PBMCs from a cancer patient that had been treated with CBD and PBMCs from healthy control.

FIG. 11A-FIG. 11C show that the function of NK cells were increased in PBMCs and CD3+ T cells isolated from splenocytes of WIN 55,212-2 treated hu-BLT mouse. PBMCs (FIG. 11A and FIG. 11B) and CD3+ T cells (FIG. 11C) were isolated from hu-BLT mice after treatment of WIN 55,212-2 (intraperitoneal (IP) injection every 2 days at 2 mg/Kg in DMSO+saline for 27 days before sacrifice) or IL-15 (IP injection of 5 μg per mice for 27 days). PBMCs were isolated from peripheral blood and treated with IL-2 (1000 u/ml) overnight before they were used in 51 Cr release assay. The Lytic units (LU) 30/10⁶ cells were determined by plotting the % cytotoxicity obtained at different effector to target ratios, and using that curve to determine the inverse number of NK cells required to lyse 30% of OSCSCs×100. The numbers of LU were then adjusted based on the % of NK cells obtained by flow cytometric analysis (FIG. 11A). PBMCs and CD3+ T cells culture supernatants were collected and IFN-γ secretions were determined using ELISA (FIG. 11B and FIG. 11C). CD3 T cells were isolated from splenocytes by negative selection of CD3+ T cells using an isolation kit (Stem cell technologies). WIN 55,212-2 treated mice showed higher NK cell mediated cytotoxicity and IFN-γ secretion in both PBMC and CD3+ T cells. IL-15 treated mouse showed higher NK cell mediated cytotoxicity in PBMC and IFN-γ secretion in CD3+ T cells.

FIG. 12A-FIG. 12C show cannabis that was administered to Patient 24: the CBD oil (FIG. 12A), CBD tincture of cannabis oils comprising the listed terpene components (FIG. 12B), and CBD oil (FIG. 12C) that was administered to Patient 24 if the CDB oil of FIG. 12A was not available.

FIG. 13A-FIG. 13D show cannabis that was administered to Patient 56: the CBD oil comprising the listed cannabinoid components (FIG. 13A), CBD oil comprising the listed terpene components (FIG. 13B), CBD bubblegum flavor comprising the listed cannabinoid components (FIG. 13C), and the THC tincture comprising the listed cannabinoid components (FIG. 13D).

FIG. 14A-FIG. 14B show cannabis that was administered to Patient p23: two types of CBD oil comprising the listed cannabinoid components (FIG. 14A and FIG. 14B).

### DETAILED DESCRIPTION OF THE INVENTION

The present invention may be understood more readily by reference to the following detailed description which forms a part of this disclosure. It is to be understood that this invention is not limited to the specific methods, products, conditions or parameters described and/or shown herein, and that the terminology used herein is for the purpose of describing particular embodiments by way of example only and is not intended to be limiting of the claimed invention.

NK cells target and kill physiologically aberrant cells, such as malignant cells and virally infected cells. NK cell cytotoxicity begins with the NK cell recognizing a target cell, which induces formation of a lytic immunological synapse (also called “lytic synapse”) at the point of contact between the NK cell and its target accompanied by a rearrangement of the actin cytoskeleton. Then, the microtubule-organizing center of the NK cell and the secretory lysosomes, which are specialized exocytic organelles found in NK cells, are polarized towards the lytic synapse. Subsequently, the secretory lysosomes bind with the plasma membrane at the lytic immunological synapse. Finally, the secretory lysosomes fuse with the plasma membrane and release their cytotoxic protein contents, primarily perforin and granzymes, to specifically kill the target cell. Perforin creates pores in the cell membrane of the target cell through which the granzymes, which are serine proteases, and associated molecules enter the cytoplasm and induce apoptosis in the target cell, thereby destroying cancerous cells and/or cells infected with viruses.

NK cells comprise about 5-15% of human peripheral blood mononuclear cells; NK cells share their role as cytotoxic and interferon-gamma (IFN-γ)-producing cells with CD8+ T cells. IFN-γ is a cytokine that has important roles in inducing and modulating an array of immune responses; IFN-γ promotes macrophage activation, mediates antiviral and antibacterial immunity, enhances antigen presentation, organizes activation of the innate immune system, coordinates lymphocyte-endothelium interaction, regulates T-helper 1 (Th1) and T-helper 2 (Th2) Th1/Th2 balance, and controls cellular proliferation and apoptosis.

It has been shown that NK cells are able to kill an average of four target cells each, but appear to become “exhausted” after this killing and have diminished perforin and granzyme B levels. Treatment with IL-2 is able to replenish the NK cells' cytotoxicity after 48 hours, apparently through increased expression of new perforin and granzymes, whose levels increase again.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

### Definitions

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. All ranges are inclusive and combinable.

The term “administering” is intended to include routes of administration which allow an agent to perform its intended function. Examples of routes of administration for treatment of a body which can be used include injection (subcutaneous, intravenous, parenteral, intraperitoneal, intratumoral, intrathecal, etc.), oral, inhalation, and transdermal routes. The injections can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier.

In some embodiments, cannabis or a cannabinoid agent can be administered intravenously, parenterally, intramuscular, subcutaneously, orally, nasally, topically, by inhalation, by implant, by injection, or by suppository. For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used. Pulmonary administration can be accomplished, for example, using any of various delivery devices known in the art such as an inhaler. See. e.g. S. P. Newman (1984) in Aerosols and the Lung, Clarke and Davis (eds.), Butterworths, London, England, pp. 197-224; PCT Publication No. WO 92/16192; PCT Publication No. WO 91/08760. For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like.

The term “altered level” refers to increased or decreased level (e.g., secretion level, expression level, activation level, activity level, etc.) of any biomarker (e.g., a biomarker indicative of immune function, e.g., a cytokine/chemokine or NK cells) as compared to the level of the said biomarker in a control sample. In some embodiments, the altered activity of e.g., a cytokine/chemokine or NK cells includes an increased or decreased activity of the cytokine/chemokine or NK cells in a sample as compared to the corresponding activity in a normal, control sample. Altered activity of the biomarker (e.g., cytokine/chemokine, e.g., IFN-γ) may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors. An altered amount or activity of a biomarker protein may be determined by detecting posttranslational modification such as phosphorylation status of the marker, which may affect the expression or activity of the biomarker protein. An altered amount or activity of a biomarker protein may be due to a differentiation state of a cancer cell. An altered amount or activity of a biomarker protein may also be due to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

The term “control” refers to any reference standard suitable to provide a comparison to the expression products in the test sample. Such a control may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In some embodiments, the control may comprise NK cells. In some embodiments, the control may comprise a blood sample comprising a cytokine/chemokine, e.g., INF-γ. In other embodiments, the control may comprise a reference standard product level from any suitable source, including but not limited to the expression level of housekeeping genes, an product level range from normal tissue (or other previously analyzed control sample), a previously determined product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In some embodiments, the control may comprise normal or non-cancerous cell/tissue/blood sample. In other embodiments, the control may comprise a product level for a set of patients, such as a set of cancer patients, or for a set of cancer patients receiving a certain treatment, or for a set of patients with one outcome versus another outcome. In other preferred embodiments, the control may comprise normal cells, cells from patients treated with combination chemotherapy, and cells from patients having benign cancer. In still other embodiments, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In other embodiments, the control comprises a ratio transformation of product levels, including but not limited to determining a ratio of product levels of two genes in the test sample and comparing it to any suitable ratio of the same two genes in a reference standard; determining product levels of the two or more genes in the test sample and determining a difference in product levels in any suitable control; and determining product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In some embodiments, the product level refers to the secretion and/or expression level of a cytokine or chemokine, e.g., IFN-γ. In some embodiments, the product level refers to the level of activity or activation of NK cells.

The amount of a biomarker (e.g., secretion level of a cytokine/chemokine, activation level of NK cells) in a cell is “significantly” higher or lower than the normal amount or the activity of the biomarker, if the amount or activity of the biomarker is greater or less, respectively, than the normal level by an amount greater than the standard error of the assay employed to assess amount, and preferably at least, about, or no more than 5%, 10%, 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the cell can be considered “significantly” higher or lower than the normal amount if the amount is at least, about, or no more than two, and preferably at least about three, four, or five times, higher or lower, respectively, than the normal amount of the biomarker. Such “significance” can also be applied to any other measured parameter described herein, such as for the level of a cytokine/chemokine (e.g., IFN-γ), activity or activation of NK cells, inhibition, cytotoxicity, cell growth, and the like.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

In some embodiments, the subject is afflicted with cancer. In various embodiments, the subject is in need of and/or benefit from the compositions and methods of the present disclosure. In various embodiments of the methods of the present invention, the subject has not undergone treatment, such as chemotherapy, radiation therapy, targeted therapy, and/or immunotherapies. In other embodiments, the subject has undergone treatment, such as chemotherapy, radiation therapy, targeted therapy, and/or immunotherapies. In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

A “therapeutically effective amount” of a substance or cells is an amount capable of producing a medically desirable result in a treated patient, e.g., decrease tumor burden, decrease the growth of tumor cells, or alleviate any symptom associated with cancer, with an acceptable benefit:risk ratio, preferably in a human or non-human mammal.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate, or stabilize the existing unwanted condition or side effects thereof). Accordingly, as used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

### NK Cells

Natural killer cells or NK cells are a type of cytotoxic lymphocyte critical to the innate immune system. The role NK cells play is analogous to that of cytotoxic T cells in the vertebrate adaptive immune response. NK cells provide rapid responses to viral-infected cells, acting at around 3 days after infection, and respond to tumor formation. Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MEW, allowing for a much faster immune reaction. They were named “natural killers” because of the initial notion that they do not require activation to kill cells that are missing “self” markers of MHC class 1. This role is especially important because harmful cells that are missing MHC I markers cannot be detected and destroyed by other immune cells, such as T lymphocyte cells.

NK cells (belonging to the group of innate lymphoid cells) are defined as large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter into the circulation. NK cells differ from natural killer T cells (NKTs) phenotypically, by origin and by respective effector functions; often, NKT cell activity promotes NK cell activity by secreting IFNγ. In contrast to NKT cells, NK cells do not express T-cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice. The NKp46 cell surface marker constitutes, at the moment, another NK cell marker of preference being expressed in both humans, several strains of mice (including BALB/c mice) and in three common monkey species.

NK cells are negatively regulated by major histocompatibility complex (MHC) class I-specific inhibitory receptors (Kane et al., 1986; Ohlen et al, 1989). These specific receptors bind to polymorphic determinants of MHC class I molecules or HLA present on other cells and inhibit NK cell lysis. In humans, certain members of a family of receptors termed killer Ig-like receptors (KIRs) recognize groups of HLA class I alleles.

KIRs are a large family of receptors present on certain subsets of lymphocytes, including NK cells. The nomenclature for KIRs is based upon the number of extracellular domains (KIR2D or KIR3D) and whether the cytoplasmic tail is either long (KIR2DL or KIR3DL) or short (KIR2DS or KIR3DS). Within humans, the presence or absence of a given KIR is variable from one NK cell to another within the NK population present in a single individual. Within the human population there is also a relatively high level of polymorphism of the KIR molecules, with certain KIR molecules being present in some, but not all individuals. Certain KIR gene products cause stimulation of lymphocyte activity when bound to an appropriate ligand. The confirmed stimulatory KIRs all have a short cytoplasmic tail with a charged transmembrane residue that associates with an adapter molecule having an immunostimulatory motif (ITAM). Other KIR gene products are inhibitory in nature.

### Therapeutic Methods

In one aspect, the present invention provides a method for increasing cytotoxicity of a natural killer (NK) cell, the method comprising administering to a subject in need thereof a composition comprising cannabis in an amount effective to induce apoptosis of a tumor cell. In certain embodiments, the method further comprises administering an amount effective to activate T cells of a composition comprising at least one of anti-CD3 antibodies, anti-CD28 antibodies, IL-2, IL-2 and anti-CD16 antibodies, and IL-2 and sAJ2 bacteria or a combination thereof. In some embodiments, administration of the composition comprising cannabis is selected from the group consisting of inhalation, oral administration, sublingual administration and topical administration. In some embodiments, the oral administration of the composition comprising cannabis comprises ingesting a cannabis oil. In other embodiments, the oral administration of the composition comprising cannabis comprises ingesting a cannabis tincture. In yet other embodiments, the oral administration of the composition comprises ingesting a food and/or beverage, wherein the food and the beverage each comprises the composition comprising cannabis. In some embodiments, the inhalation is by smoking or vaporizing.

In certain embodiments of the provided methods, the composition comprising the at least one of anti-CD3 antibodies, anti-CD28 antibodies, IL-2, IL-2 and anti-CD16 antibodies, and IL-2 and sAJ2 bacteria is administered intravenously. In various embodiments, each of the anti-CD3 antibodies, anti-CD28 antibodies, and anti-CD16 antibodies are monoclonal antibodies. In some embodiments, the composition comprising cannabis and the composition comprising at least one of anti-CD3 antibodies, anti-CD28 antibodies, IL-2, IL-2 and anti-CD16 antibodies, and IL-2 and sAJ2 bacteria are administered concurrently or sequentially. In particular embodiments, the subject has cancer and/or a tumor.

In another aspect, the present invention provides a method for increasing or restoring secretion of interferon gamma (IFN-γ) by a peripheral blood mononuclear cell (PMBC), the method comprising administering to a subject in need thereof a composition comprising cannabis. In some embodiments, the PMBC is a natural killer (NK) cell, a natural killer T (NKT), a CD4 T helper (TH) lymphocyte, and/or a CD8 cytotoxic cell. In particular embodiments, the PMBC is a natural killer (NK) cell. In certain embodiments, the composition comprising cannabis is administered in an amount effective to reactivate an exhausted NK cell. In certain embodiments, the method further comprises administering an amount effective to activate T cells of a composition comprising at least one of anti-CD3 antibodies, anti-CD28 antibodies, IL-2, IL-2 and anti-CD16 antibodies, and IL-2 and sAJ2 bacteria or a combination thereof.

In some embodiments of the provided methods, administration of the composition comprising cannabis is selected from the group consisting of inhalation, oral administration, sublingual administration and topical administration. In particular embodiments, the oral administration of the composition comprising cannabis comprises ingesting a cannabis oil. In some embodiments, the oral administration of the composition comprising cannabis comprises ingesting a cannabis tincture. In other embodiments, the oral administration of the composition comprises ingesting a food and/or beverage, wherein the food and the beverage each comprises the composition comprising cannabis. In some embodiments, the inhalation is by smoking or by vaporizing.

In additional embodiments, the composition comprising the at least one of anti-CD3 antibodies, anti-CD28 antibodies, IL-2, IL-2 and anti-CD16 antibodies, and IL-2 and sAJ2 bacteria is administered intravenously. In some embodiments, each of the anti-CD3 antibodies, anti-CD28 antibodies, and anti-CD16 antibodies are monoclonal antibodies. In various embodiments, the composition comprising cannabis and the composition comprising the at least one of anti-CD3 antibodies, anti-CD28 antibodies, IL-2, IL-2 and anti-CD16 antibodies, and IL-2 and sAJ2 bacteria are administered concurrently or sequentially. In particular embodiments, the subject has cancer and/or a tumor. In certain embodiments, the subject demonstrates decreased secretion of IFN-γ prior to the administration of the composition comprising cannabis.

In a further aspect, the present invention provides a method for preventing or reversing inactivation of cytotoxicity of a natural killer (NK) cell, the method comprising administering to a subject in need thereof a composition comprising cannabis in an amount effective to reactivate an exhausted NK cell. In some embodiments, the method further comprises administering an amount effective to activate T cells of a composition comprising at least one of anti-CD3 antibodies, anti-CD28 antibodies, IL-2, IL-2 and anti-CD16 antibodies, and IL-2 and sAJ2 bacteria. In other embodiments, administration of the composition comprising cannabis is selected from the group consisting of inhalation, oral administration, sublingual administration and topical administration.

In additional embodiments, the oral administration of the composition comprising cannabis comprises ingesting a cannabis oil. In further embodiments, the oral administration of the composition comprising cannabis comprises ingesting a cannabis tincture. In some embodiments, the oral administration of the composition comprises ingesting a food and/or beverage, wherein the food and the beverage each comprises the composition comprising cannabis. In some embodiments, the inhalation is by smoking or vaporizing.

In some embodiments, the composition comprising at least one of anti-CD3 antibodies, anti-CD28 antibodies, IL-2, IL-2 and anti-CD16 antibodies, and IL-2 and sAJ2 bacteria is administered intravenously. In various embodiments, each of the anti-CD3 antibodies, anti-CD28 antibodies, and anti-CD16 antibodies are monoclonal antibodies. In other embodiments, the composition comprising cannabis and the composition comprising at least one of anti-CD3 antibodies, anti-CD28 antibodies, IL-2, IL-2 and anti-CD16 antibodies, and IL-2 and sAJ2 bacteria are administered concurrently or sequentially. In specific embodiments, the subject has cancer and/or a tumor.

In another aspect, the present invention provides a method for treating a subject having cancer or a tumor, the method comprising: (a) obtaining a blood or tissue sample of the subject; (b) determining a level of activation of an NK cell and/or a level of secretion of IFN-γ by a NK cell in the blood or tissue sample of the subject; (c) comparing the determined level of activation of the NK cell and/or the determined level of secretion of IFN-γ by the NK cell to a level of activation of a NK cell and/or a level of secretion of IFN-γ by a NK cell in the blood or tissue sample of a control healthy subject; wherein when the determined level of activation of a NK cell and/or the determined level of secretion of IFN-γ by the NK cell in the blood or tissue sample of the subject is decreased compared to the level of activation of a NK cell and/or the level of secretion of IFN-γ by a NK cell in the blood or tissue sample of the control healthy subject, (d) administering to the subject an amount effective to induce apoptosis of a cancer cell of a composition comprising cannabis.

In some embodiments, the methods provided herein further comprise administering an amount effective to activate T cells of a composition comprising at least one of anti-CD3 antibodies, anti-CD28 antibodies, IL-2, IL-2 and anti-CD16 antibodies, and IL-2 and sAJ2 bacteria. In some embodiments of the herein provided methods, administration of the composition comprising cannabis is selected from the group consisting of inhalation, oral administration, sublingual administration and topical administration. In certain embodiments, the oral administration of the composition comprising cannabis comprises ingesting a cannabis oil. In additional embodiments, the oral administration of the composition comprising cannabis comprises ingesting a cannabis tincture. In other embodiments, the oral administration of the composition comprises ingesting a food and/or beverage, wherein the food and the beverage each comprises the composition comprising cannabis.

In some embodiments, the inhalation is by smoking or vaporizing. In particular embodiments, the composition comprising at least one of anti-CD3 antibodies, anti-CD28 antibodies, IL-2, IL-2 and anti-CD16 antibodies, and IL-2 and sAJ2 bacteria is administered intravenously. In some embodiments, each of the anti-CD3 antibodies, anti-CD28 antibodies, and anti-CD16 antibodies are monoclonal antibodies. In other embodiments, the composition comprising cannabis and the composition comprising at least one of anti-CD3 antibodies, anti-CD28 antibodies, IL-2, IL-2 and anti-CD16 antibodies, and IL-2 and sAJ2 bacteria are administered concurrently or sequentially.

### Cannabis and Cannabinoid Compositions

In certain aspects, provided herein is a method for the treatment of cancer and/or tumors is provided comprising administering a therapeutically effective amount of a composition comprising cannabis as described herein, to a subject in need thereof.

The Cannabis genus of plants comprise the species Cannabis sativa; the plant is commonly known as hemp. Two additional species, C. indica and C. rideralis are considered as subspecies of C. sativa. The cannabis plant synthesizes over 100 chemical compounds known as phytocannabinoids, including cannabinoid acids and terpenes (terpenoids). The predominant cannabinoid acids produced are THCA (Δ⁹-tetrahydrocannabinolic acid, referred to herein as “THC”), CBGA (Cannabigerolic acid), CBDA (Cannabidiolic acid), CBCA (Cannabichromenenic acid), CBGVA (Cannabigerovarinic acid), THCVA (Tetrahydrocanabivarinic acid), CBDVA (Cannabidivarinic acid) and CBCVA (Cannabichromevarinic acid), of which THCA and CBDA are usually the most abundant. These cannabinoid acids must be decarboxylated, generally by heat, to yield the corresponding cannabinoids; thus, CBDA and THCA are decarboxylated to CBD (Cannabidiol) and THC (Δ⁹-tetrahydrocannabinol), respectively. THC is the cannabinoid with psychoactive properties; CBD alone has no psychotropic effects. CBD and/or the terpenoids are thought acts as entourage compound(s) to reduce THC side effects of therapeutic cannabis formulations.

Cannabis has been used for palliative care of cancer patients to reduce symptoms of pain, chemotherapy-induced nausea and vomiting, sleep disorders, anxiety, and depression. Studies suggest that cannabinoids, such as THC, demonstrate anti-tumor effects in experimental models of cancer. Some cannabinoid receptor agonists, such as the synthetic cannabinoid receptor agonists WIN 55,212-2 or JWH-133, promote cancer cell death. Other studies have found that the endocannabinoid system might be overactivated in cancer and have proposed a pro-tumorigenic effect of cannabinoids. How cannabinoids produce an anti-tumor effects, including binding to various receptors, has not been established.

Accordingly, as used herein, the term “cannabis” includes but is not limited to the cannabis genus of plants, its derivatives, extracts, and isolates, any component thereof, at least one cannabinoid agent (e.g., agonist, synthetic or natural), or any combination thereof.

In some embodiments, the composition comprising cannabis is formulated as a pharmaceutical composition for inhalation. In other embodiments, the composition comprising cannabis is formulated as a pharmaceutical composition for oral administration. In additional embodiments, the composition comprising cannabis is formulated as a pharmaceutical composition for sublingual administration. In further embodiments, the composition comprising cannabis is formulated as a pharmaceutical composition for topical administration. In some embodiments, the cannabis composition comprises cannabis oil, which is prepared as described herein. In other embodiments, the cannabis composition comprises a cannabis tincture.

In particular embodiments, the composition comprising cannabis comprises a therapeutically effective amount of the cannabis oil. In certain embodiments, the composition comprising cannabis comprises a therapeutically effective amount of the cannabis oil effective to induce apoptosis of the target cell, wherein the target cell is a cancer cell and/or a tumor cell. In certain embodiments, the apoptosis of the target cell is induced by NK cell release of cytotoxic granules containing perform and granzymes, which leads to lysis of the target cells.

In some embodiments, the therapeutically effective amount of the cannabis oil is effective to increase NK cell release of cytotoxic granules containing perforin and granzymes at (a) a level that is greater than a level of NK cell release of cytotoxic granules in a healthy cell (control), and (b) a level that is greater than at a level of NK cell release of cytotoxic granules in a patient having cancer and/or a tumor, wherein the patient has not been administered a composition comprising cannabis.

In other embodiments, the composition comprising cannabis comprises a therapeutically effective amount of the cannabis oil effective to increase or restore secretion of interferon gamma (IFN-γ) by a peripheral blood mononuclear cell (PMBC). In specific embodiments, the PMBC cell is an NK cell, wherein the increase in or restoration of secretion of IFN-γ prevents or reverses inactivation of cytotoxicity of the NK cell. In other embodiments, the composition comprising cannabis comprises a therapeutically effective amount of the cannabis oil effective to reactivate an exhausted NK cell.

It will be appreciated that the compounds and compositions, according to the methods of the present invention, may be administered using any amount and any route of administration effective for the treatment of cancer and/or tumors in which a composition comprising cannabis has a therapeutically useful role. Thus, the expression “therapeutically effective amount” as used herein, refers to a sufficient amount of a composition comprising cannabis to induce an NK cell to increase its cytotoxicity and to induce increased secretion of IFN-γ and to exhibit a therapeutic effect. The exact amount required will vary from subject to subject, depending on the species, age, and general condition of the subject, the severity of the infection, the particular therapeutic agent, its mode and/or route of administration, and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, of the compositions comprising cannabis oil, the therapeutically effective amount of the cannabis oil (also called a CBD tincture of cannabis oils) comprises a 0.25 ml per dose of 85% CBD oil and a 0.13 ml per dose of 50% THC oil, with each of the CBD oil and the THC oil administered three times per day to the patient in need thereof. (Example 11). In other embodiments, of the compositions comprising cannabis oil, the therapeutically effective amount of the cannabis oil or cannabis tincture comprises 148 mg of CBD and 65 mg of THC, and the CBD and THC are every eight hours, respectively. (Example 11). In further embodiments, the therapeutically effective amount of the cannabis oil or cannabis tincture comprises about 72.58% total mass of CBD and about 2.95% total mass of THC. (Example 11). In some embodiments, the CBD oil comprises the following cannabinoids: delta9-THC, CBD, CBDV, CBG, and CBC.

In other embodiments, the CBD tincture of cannabis oils comprises the following terpene components: beta-Caryophyllene, alpha-Humulene, cis-Nerolidol, trans-Nerolidol, alpha-Bisabolol, Guaiol, Caryophyllene oxide, beta-Myrcene, alpha-Terpinene and combinations thereof. In some embodiments, the therapeutically effective amount of the cannabis oil or cannabis tincture comprises a 0.072% total mass of terpene. (Example 11). In some embodiments, the THC oil comprises the following cannabinoid components: delta9-THC, delta9-THCA, CBD, CBDA, CBG, CBN, CBC, and delta8-THC, wherein the delta9-THC Max. comprises 52.2 wt. % of the cannabinoids in the THC oil or 522.04 mg/ml; the CBD Max. comprises 7.67 wt. % of the cannabinoids in the THC oil or 76.71 mg/gram; and the CBG Max. comprises 2.51 wt. % of the cannabinoids in the THC oil or 25.13 mg/gram. (Example 11). In some embodiments, the THC tincture of cannabis oils comprises the following terpene components: alpha-Bisabolol, beta-Caryophyllene, Caryophyllene oxide, alpha-Humulene, Limonene, Linalool, Myrcene, alpha-Pinene, beta-Pinene and Terpinolene and combinations thereof. In some embodiments, the therapeutically effective amount of the cannabis oil or cannabis tincture comprises 11.22 mg/g of terpene. (Example 11). In additional embodiments, the therapeutically effective amount of the CBD tincture of cannabis oils comprises the following terpene components: alpha-Bisabolol, beta-Caryophyllene, Caryophyllene oxide, alpha-Humulene, limonene, linalool, mycerene, alpha-pinene, beta-pinene, and terpinolene and combinations thereof. In some embodiments, the therapeutically effective amount of the cannabis oil or cannabis tincture comprises 29.69 mg/g of terpenes.

In other embodiments, the therapeutically effective amount of the cannabis oil or cannabis tincture comprises a 85.84% total mass of CBD and a 4.19% total mass of THC. In some embodiments, the CBD oil comprises the following cannabinoids: delta9-THC, CBD, CBDV, CBG, and CBC. In some embodiments, the CBD tincture of cannabis oils comprises the following terpene components: alpha-Bisabolol, beta-Caryophyllene, Caryophyllene oxide, alpha-Humulene, Limonene, Linalool, Myrcene, alpha-Pinene, beta-Pinene and Terpinolene and combinations thereof. In some embodiments, the therapeutically effective amount of the cannabis oil or cannabis tincture comprises 26.69 mg/g of terpene. (Example 11).

In some embodiments, the therapeutically effective amount of the cannabis oil a 50 mg/ml CBD tincture comprising the following cannabinoid components: delta9-THC total (0.24 wt. %), CBD (total 5.50 wt. %) and CBG (Total 0.02 wt %). (Example 11). In additional embodiments, the CBD tincture of cannabis oils comprises the following terpene components: alpha-Bisabolol, beta-Caryophyllene, Caryophyllene oxide, alpha-Humulene, Limonene, Linalool, Myrcene, alpha-Pinene, beta-Pinene and Terpinolene and combinations thereof. In some embodiments, the therapeutically effective amount of the cannabis oil or cannabis tincture comprises 26.80 mg/g of terpene. (Example 11). In further embodiments, the administered THCA oil comprises the following cannabinoid components: THCA, delta9-THC, CBGa, CBG, CBC, CBD, CBDV, CBN, THCC, delta8-THC. In some embodiments, the therapeutically effective amount of the THCA oil comprises 231.90 mg/unit THC total and 0.44 mg/unit CBD total. (Example 11). In some embodiments, the THCa tincture of cannabis oils comprises a combination of terpene components in which myrcene, pinene, and caryophyllene comprises the majority of terpenes and in which terpenes comprise 89.766 wt. % of the THCa tincture of cannabis oils, as shown in Example 11. Further exemplary embodiments of cannabis oils, THC oils, CBDA oils, and TCHA oils are set forth in Example 11.

In some embodiments, a patient administered the above-described therapeutically effective amounts of the cannabis oil has a reduction of tumor size, an elimination of a tumor, eradication of cancer, i.e., the patient is cancer-free, a reduction in pain and/or fever caused by cancer, a reduction in nausea and/or vomiting from chemotherapy, an increase in appetite, better sleep, and/or increase in weight.

In further embodiments, the cannabis oils comprise a flavoring, such as a natural and/or an artificial flavoring, including but not limited to a fruit flavor extract, vanilla, cinnamon, almond and chocolate flavors and candy or chewing gum flavors, including bubblegum flavor. The fruit flavors include but not limited to grape, cranberry, apple, strawberry and banana, pineapple and coconut, lemon-lime, cherry, flavors and combinations thereof.

Furthermore, after formulation with a pharmaceutically acceptable carrier in a desired dosage, the pharmaceutical compositions of this invention can be administered to humans and other animals orally, rectally, parenterally, intracisternally, intravaginally, intraperitoneally, subcutaneously, intradermally, intra-ocularly, topically (as by powders, ointments, or drops), buccally, as an oral or nasal spray, or the like, depending on the severity of the disease or disorder being treated. In some embodiments, pharmaceutically-acceptable carriers and/or diluents and/or adjuvants (collectively referred to herein as “carrier” materials) are added to the composition comprising cannabis oil.

In certain embodiments, the at least one compound (e.g., cannabis or at least one component thereof; e.g., CBD, THC, or a combination thereof) of the present disclosure may be administered to a subject at dosage levels of at least, about, or no more than 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, 0.3 mg/kg, 0.31 mg/kg, 0.32 mg/kg, 0.33 mg/kg, 0.34 mg/kg, 0.35 mg/kg, 0.36 mg/kg, 0.37 mg/kg, 0.38 mg/kg, 0.39 mg/kg, 0.4 mg/kg, 0.41 mg/kg, 0.42 mg/kg, 0.43 mg/kg, 0.44 mg/kg, 0.45 mg/kg, 0.46 mg/kg, 0.47 mg/kg, 0.48 mg/kg, 0.49 mg/kg, 0.5 mg/kg, 0.51 mg/kg, 0.52 mg/kg, 0.53 mg/kg, 0.54 mg/kg, 0.55 mg/kg, 0.56 mg/kg, 0.57 mg/kg, 0.58 mg/kg, 0.59 mg/kg, 0.6 mg/kg, 0.61 mg/kg, 0.62 mg/kg, 0.63 mg/kg, 0.64 mg/kg, 0.65 mg/kg, 0.66 mg/kg, 0.67 mg/kg, 0.68 mg/kg, 0.69 mg/kg, 0.7 mg/kg, 0.71 mg/kg, 0.72 mg/kg, 0.73 mg/kg, 0.74 mg/kg, 0.75 mg/kg, 0.76 mg/kg, 0.77 mg/kg, 0.78 mg/kg, 0.79 mg/kg, 0.8 mg/kg, 0.81 mg/kg, 0.82 mg/kg, 0.83 mg/kg, 0.84 mg/kg, 0.85 mg/kg, 0.86 mg/kg, 0.87 mg/kg, 0.88 mg/kg, 0.89 mg/kg, 0.9 mg/kg, 0.91 mg/kg, 0.92 mg/kg, 0.93 mg/kg, 0.94 mg/kg, 0.95 mg/kg, 0.96 mg/kg, 0.97 mg/kg, 0.98 mg/kg, 0.99 mg/kg, 1 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 61 mg/kg, 62 mg/kg, 63 mg/kg, 64 mg/kg, 65 mg/kg, 66 mg/kg, 67 mg/kg, 68 mg/kg, 69 mg/kg, 70 mg/kg, 71 mg/kg, 72 mg/kg, 73 mg/kg, 74 mg/kg, 75 mg/kg, 76 mg/kg, 77 mg/kg, 78 mg/kg, 79 mg/kg, 80 mg/kg, 81 mg/kg, 82 mg/kg, 83 mg/kg, 84 mg/kg, 85 mg/kg, 86 mg/kg, 87 mg/kg, 88 mg/kg, 89 mg/kg, 90 mg/kg, 91 mg/kg, 92 mg/kg, 93 mg/kg, 94 mg/kg, 95 mg/kg, 96 mg/kg, 97 mg/kg, 98 mg/kg, 99 mg/kg, or 100 mg/kg. In some embodiments, the at least one compound of the present invention is administered to the subject at least, about, or no more than once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the at least one compound of the present invention is administered to the subject at least, about, or no more than 1, 2, 3, 4, 5, 6, 7, 8, 9, or 10 times a day. In addition, due to the safety of the compounds, the treatment for indefinite amount of time is also contemplated herein.

In certain embodiments, the compounds of the invention may be administered at dosage levels of about 0.001 mg/kg to about 50 mg/kg, preferably from about 0.1 mg/kg to about 10 mg/kg for parenteral administration, or preferably from about 1 mg/kg to about 50 mg/kg, more preferably from about 10 mg/kg to about 50 mg/kg for oral administration, of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally. Exemplary dosing regimens are presented in, e.g., Example 11 of the present disclosure.

In each of the above-described embodiments, the composition comprising cannabis may further comprise a plant-based terpenoid. In particular embodiments, the plant-based terpenoid is a cannabis terpenoid selected from the group consisting of a limonene, myrcene, α-pinene, linalool, (3-caryophyllene, caryophyllene oxide, nerolidol, phytol and combinations thereof.

Further provided herein is a composition comprising a cannabinoid (e.g., cannabinoid agonist) for a treatment of cancer. In some embodiments, the composition further comprises the cannabinoid agonist which is provided in an amount of between about 0.01 and 1000 mg for dosing. In some embodiments, the administration of the composition is selected from inhalation, parenteral administration, oral administration, sublingual administration, and topical administration. In some embodiments, the cannabinoid is WIN 55,212-2. In certain embodiments, the composition also comprises a cancer therapy, e.g., a DNA-interacting agent, an antimetabolite, a tubulin-interacting agent, a molecular-targeted therapeutic agent, an epigenetic-action inhibitor, a hormone, an immunotherapy, and/or another cannabinoid agent/cannabis.

In certain embodiments, said treatment causes the cancer to enter a state of static growth. In some embodiments, said treatment causes cancer cell death. In certain embodiments, the cell death is autophagic, apoptotic, or necrotic.

Non-limiting examples of cannabinoid agonists include, CP-55,940, WIN 55,212-2, JWH-015, JWH-133, SR141716 (rimonabant), SR144528, and ACEA.

CP 55,940 is a cannabinoid which mimics the effects of naturally occurring tetrahydrocannabinol (THC) (a cannabinoid). The molecular weight is 376.6, and the its chemical name is (−)-cis-3-[2-Hydroxy-4-(1,1-dimethylheptyl)phenyl]-trans-4-(3-hydroxypropyl)cyclohexanol.

WIN 55,212-2 is a chemical described as an aminoalkylindole derivative, which produces effects similar to those of cannabinoids such as THC but has an entirely different chemical structure. The molecular weight is 426.5, and its chemical name is (R)-(+)-[2,3-Dihydro-5-methyl-3-(4-morpholinylmethyl)pyrrolo[1,2,3-de]-1,4-benzoxazin-6-yl]-1-naphthalenylmethanone mesylate.

JWH-015 is a chemical from the naphthoylindole family that acts as a subtype-selective cannabinoid agonist. The molecular weight is 327.4, and its chemical name is (2-methyl-1-propyl-1H-indol-3-yl)-1-naphthalenyl-methanone.

JWH 133 is a synthetic cannabinoid (CB) that is a subtype-selective cannabinoid agonist. Its molecular weight is 312.5, and its chemical name is 3-(1,1-dimethylbutyl)-6aR,7,10,10aR-tetrahydro-6,6,9-trimethyl-6H-dibenzo[b,d]pyran.

SR141716 (rimonabant) is an anorectic antiobesity drug that is a subtype-selective cannabinoid inverse agonist. Its molecular weight is 463.8, and its chemical name is 5-(4-chlorophenyl)-1-(2,4-dichlorophenyl)-4-methyl-N-1-piperidinyl-1H-pyrazole-3-carboxamide.

SR144528 is a drug that acts as a potent and highly subtype-selective cannabinoid inverse agonist. The molecular weight is 476 and its chemical structure is 5-(4-chloro-3-methylphenyl)-1-[(4-methylphenyl)methyl]-N-[(1S,2S,4R)-1,3,3-trimethylbicyclo[2.2.1]hept-2-yl]-1H-pyrazole-3-carboxamide.

Arachidonyl-2′-chloroethylamide (ACEA) is a synthetic subtype-specific cannabinoid agonist. Its molecular weight is 366, and its chemical structure is N-(2-chloroethyl)-5Z,8Z,11Z,14Z-eicosatetraenamide.

The dosage of the cannabinoid agonist, or a derivative thereof, administered to a patient may vary and may be an amount of from about 0.2 mg/kg to about 50 mg/kg, based on the weight of the patient. Thus, the dosage of the cannabinoid, or a derivative thereof, may vary depending upon, inter alia, nature of the disorder, the sex of the patient, i.e., male or female, etc. and may be about 0.2-50 mg/kg, about 1-45 mg/kg, about 10-40 mg/kg, about 20-40 mg/kg, about 25-35 mg/kg, based on the weight of the patient. The dosage of the cannabinoid, or a derivative thereof, may vary depending upon, inter alia, the severity of the disorder, the nature of the disorder, the sex of the patient, i.e., male or female, etc. and may be about 1 μmol (about 2.3 mg in the case of WIN55,212-2), about 10 μmol (about 23 mg), about 20 μmol (about 47 mg), about 30 μmol (about 70 mg), about 40 μmol (about 93 mg), about 45 μmol (about 105 mg), about 50 μmol (about 117 mg), about 55 μmol (about 129 mg), about 60 μmol (about 141 mg), about 65 μmol (about 152 mg), about 70 μmol (about 164 mg), about 75 μmol (about 176 mg), about 80 μmol (about 187 mg), about 85 μmol (about 200 mg), about 90 μmol (about 211 mg), about 95 μmol (about 223 mg), or about 100 μmol (about 234 mg). Other cannabinoids may be provided at the corresponding amounts.

The effective amounts of compound or drug can and will vary according to the specific composition being utilized, the mode of administration and the age, weight and condition of the subject. Dosages for a particular individual subject can be determined by one of ordinary skill in the art using conventional considerations. In general, the amount of cannabinoid agent will be between about 0.01 to about 1000 milligrams per day and more typically, between about 0.5 to about 750 milligrams per day and even more typically, between about 1.0 to about 500 milligrams per day, between about 1.0 to about 100 milligrams per day, between about 5.0 to about 100 milligrams per day, and between about 20.0 to about 100 milligrams per day. The daily dose can be administered in one, two, three or four doses per day.

It will be understood by the person skilled in the art that the dosage regimen and the frequency of administration may be tailored depending upon, inter alia, the severity of the disorder, the nature of the disorder, the sex of the patient, i.e., male or female, etc. and may be for example, generally based on a dose regime of once weekly, twice weekly, three times weekly, four times weekly, five times weekly, six times weekly, every day, or multiple times a day; for one week in a 3-week cycle. Alternatively, the dosage regime may be generally based on a dose regime of once weekly, twice weekly, three times weekly, four times weekly, five times weekly, six times weekly, every day, or multiple times a day; for two weeks in a 3-week cycle.

Alternatively, the dosage regime may be generally based on a dose regime of once weekly, twice weekly, three times weekly, four times weekly, five times weekly, six times weekly, every day, or multiple times a day; for 3 weeks in a 3-week cycle. Alternatively, the dosage regime may be generally based on a dose regime of once weekly, twice weekly, three times weekly, four times weekly, five times weekly, six times weekly, every day, or multiple times a day; for one week in a 4-week cycle. Alternatively, the dosage regime may be generally based on a dose regime of once weekly, twice weekly, three times weekly, four times weekly, five times weekly, six times weekly, every day, or multiple times a day; for two weeks in a 4-week cycle. Alternatively, the dosage regime may be generally based on a dose regime of once weekly, twice weekly, three times weekly, four times weekly, five times weekly, six times weekly, every day, or multiple times a day; for 3 weeks in a 4-week cycle. Alternatively, the dosage regime may be generally based on a dose regime of once weekly, twice weekly, three times weekly, four times weekly, five times weekly, six times weekly, every day, or multiple times a day; for 4 weeks in a 4-week cycle.

When the cannabinoid agonist, or a derivative thereof, is administered by way of infusion, the duration of the infusion may vary. Thus, the infusion may be administered as an intravenous infusion over a period of 15 minutes, 30 minutes, 45 minutes, 1 hour, 1.5 hours, 2 hours, 2.5 hours, 3 hours, 3.5 hours, 4 hours, 4.5 hours, 5 hours, 5.5 hours, or 6 hours, each treatment day during a cycle. The dosing may be once a day. The dosing can also be multiple times a day. The dose can be q.d. (once a day), t.i.d. (three times a day), q.i.d. (four times a day), q4h, q3h, q2h, and q1h.

A subject may be administered with the composition of the present disclosure over the lifetime of the subject, e.g., for prevention of cancer or treatment of a chronic condition. The subject may be administered with the composition until the symptoms resolve. The subject may continue to be administered with the composition until the cancer is no longer seen by biopsy or other relevant diagnostic measures. The dose regimen can be altered throughout the treatment period. The dose regimen can be altered, e.g., reduced in the amount of cannabis or frequency of dosing, if the cancer cells stop growing. It may also be tapered off to zero or a maintenance dose if the cancer has gone into remission, stopped growing, or otherwise become benign.

### Pharmaceutical Compositions

Generally speaking, the pharmacokinetics of the particular agent to be administered will dictate the most preferred method of administration and dosing regimen. The cannabis or cannabinoid agent can be administered as a pharmaceutical composition with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17th Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation.

Moreover, the cannabis or cannabinoid agent can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, or magnesium carbonate.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavoring agent such as peppermint, oil of Wintergreen, or cherry flavoring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound(s) may be incorporated into sustained-release preparations and formulations, including those that allow specific delivery of the active peptide to specific regions of the gut.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Pharmaceutically acceptable carriers and/or diluents include any and all solvents, dispersion media, coatings, antibacterial and antifungal agents, isotonic and absorption delaying agents and the like.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the active ingredients may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension.

In the case of a spray, this may be achieved for example by means of a metering atomizing spray pump. To improve nasal delivery and retention the compounds according to the invention may be encapsulated with cyclodextrins or formulated with other agents expected to enhance delivery and retention in the nasal mucosa.

Administration to the respiratory tract may also be achieved by means of an aerosol formulation in which the active ingredient is provided in a pressurized pack with a suitable propellant such as a chlorofluorocarbon (CFC) for example dichlorodifluoromethane, trichlorofluoromethane, or dichlorotetrafluoroethane, carbon dioxide, or other suitable gas.

The aerosol may conveniently also contain a surfactant such as lecithin. The dose of drug may be controlled by provision of a metered valve.

Alternatively, the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier may form a gel in the nasal cavity. The powder composition may be presented in unit dose form for example in capsules or cartridges of, e.g. gelatin, or blister packs from which the powder may be administered by means of an inhaler.

Additional embodiments of pharmaceutical compositions are provided below. As used herein the pharmaceutically acceptable carrier is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds can also be incorporated into the compositions.

A pharmaceutical composition of the present invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Inhibition of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds including, e.g., cannabis or cannabinoids may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, cannabis or cannabinoids are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present invention are dictated by, and directly dependent on, the unique characteristics of the active compound, the particular therapeutic effect to be achieved, and the limitations inherent in the art of compounding such an active compound for the treatment of individuals.

### Exemplary Diseases

When the use herein described comprises the treatment of cancer, the cancer may be selected from one or more of primary cancer, breast cancer, colon cancer, prostate cancer, non-small cell lung cancer, glioblastoma, lymphoma, melanoma, mesothelioma, liver cancer, intrahepatic bile duct cancer, oesophageal cancer, pancreatic cancer, stomach cancer, laryngeal cancer, brain cancer, ovarian cancer, testicular cancer, cervical cancer, oral cancer, pharyngeal cancer, renal cancer, thyroid cancer, uterine cancer, urinary bladder cancer, hepatocellular carcinoma, thyroid carcinoma, osteosarcoma, small cell lung cancer, leukaemia, myeloma, gastric carcinoma and metastatic cancers.

Cancer, tumor, or hyperproliferative disorder refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

### Combination Therapy/Cancer Therapy

In some embodiments, a cannabis of the present disclosure is administered conjointly with an additional therapy. In some embodiments, the additional therapy is a cancer therapy. In some embodiments, the pharmaceutical composition further comprises an additional therapy (e.g., cancer therapy) other than cannabis of the present disclosure. Any suitable additional therapy may be used provided that the activity of the additional therapy and/or the cannabis is not grossly diminished when combined. In other embodiments, an additional therapy is not part of the pharmaceutical composition comprising cannabis but is nonetheless administered conjointly to a subject.

The therapeutic agents of the present invention can be used alone or can be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, agents of the present invention can be administered with a therapeutically effective dose of chemotherapeutic agent.

In other embodiments, agents of the present invention are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

Suitable anti-cancer drugs include trastuzumab or protein tyrosine kinase inhibitors (e.g. lapatinib). In some embodiments, the subject has previously been administered, or is currently being administered, an aromatase inhibitor. In some embodiments, the aromatase inhibitor is selected from aminoglutethimide, testolactone, anastrozole, letrozole, exemestane, vorozole, formestane, megestrol acetate, and fadrozole. In some embodiments the anti-cancer drug is a hormone agonist or antagonist. In some embodiments the hormone antagonist or hormone agonist is an ER antagonist. Non-limiting exemplary ER antagonists include tamoxifen and fulvestrant or a combination thereof.

In some embodiments, the cancer therapy is a selective estrogen receptor modulator. Selective estrogen receptor modulators are a class of medicines that act upon the estrogen receptor. Their action is different in various tissues, thereby granting the possibility to selectively inhibit or stimulate estrogen-like action in various tissues. Selective estrogen receptor modulators include: afimoxifene (4-hydroxytamoxifen), arzoxifene, bazedoxifene, clomifene, lasofoxifene, ormeloxifene, ormeloxifene, raloxifene, tamoxifen, or toremifene and they are used for a variety of medical indications.

Some selective estrogen receptor modulators used as anti-tumoral agents include raloxifene, tamoxifen, or toremifine.

In alternative embodiments, the cancer therapy may be an alkylating agent. An alkylating agent is a type of anti-neoplastic agent that works by interfering with DNA in several ways. Alkyl groups, are added to DNA, which causes the cell to degrade the DNA as the cell tries to replace them. Alkylating agents also interfere with the bonds between DNA strands, preventing the DNA from separating, which is a step required in DNA replication. Also, the alkylating agents can create mismatching DNA-base pairs by converting one DNA base into a different one.

All these changes occur when a cell is preparing to divide, and the permanent damage they cause results in cessation of division and cell death.

Preferably the alkylating agent is selected from the group consisting of: alkyl sulfonates, busulfan, ethyleneimines and methylmelamines, hexamethymelamine, altretamine, thiotepa, nitrogen mustards, cyclophosphamide, mechlorethamine, mustine, uramustine, uracil mustard, melphalan, chlorambucil, ifosfamide, nitrosureas, carmustine, cisplatin, streptozocin, triazenes, dacarbazine, imidazotetrazines, and temozolomide. Alkylating agents used as anti-tumoral agents include cisplatin, temozolamide, and carmustine.

Antimetabolites are only similar to normal metabolites found within the cell. When cells incorporate an antimetabolite into their cellular metabolism, the proper functioning of the cell is interfered with, usually preventing the cell from dividing. Antimetabolites interfere with specific phases of the cell-cycle. Antimetabolites are classified according to the substances with which they interfere, i.e., they antagonize or inhibit folic acid, pyrimidine, purine, and adenosine deaminase. Examples include: Folic acid antagonist: methotrexate; pyrimidine antagonists: 5-Fluorouracil, 5-fluorodeoxyuridine, cytosine arabinoside, capecitabine, and gemcitabine; purine antagonists: 6-Mercaptopurine and 6-Thioguanine; adenosine deaminase inhibitors: 2-chloro-2′-deoxyadenosine, fludarabine and pentostatin.

In any of the embodiments described herein, the cannabis and the one or more other agents among those described herein may be combined into a single dosage unit, or they may be administered in separate dosage units at the same time or at different times.

In some embodiments, the cancer therapy is an immunotherapy. Immunotherapy is a targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF is known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy also encompasses immune checkpoint modulators. Immune checkpoints are a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments, the cancer vaccine is administered in combination with one or more inhibitors of immune checkpoints, such as PD1, PD-L1, and/or CD47 inhibitors.

Adoptive cell-based immunotherapies can be combined with the therapies of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (MET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like.

In other embodiments, immunotherapy comprises non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In some embodiments, immunomodulatory cytokines, such as interferons, G-CSF, imiquimod, TNFalpha, and the like, as well as modulators thereof (e.g., blocking antibodies or more potent or longer lasting forms) are used. In some embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In some embodiments, immunomodulatory chemokines, such as CCL3, CCL26, and CXCL7, and the like, as well as modulators thereof (e.g., blocking antibodies or more potent or longer lasting forms) are used. In some embodiments, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C(indole-3-carbinol)/DIM(di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.
Similarly, agents and therapies other than immunotherapy or in combination thereof can be used with in combination with an anti-KHK antibodies to treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

In some embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In other embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1, 8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of beta-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In other embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

In some embodiments, probiotics are used conjointly with the composition comprising cannabis of the present disclosure.

### Probiotic Bacteria

In some embodiments, the composition of the present disclosure further comprises at least one probiotic bacterial strain, capable of regulating NK cell function. Such probiotic bacteria induce significant split anergy in activated NK cells, leading to a significant induction of IFN-γ and TNF-α. In addition, such probiotic bacteria induce significant expansion of NK cells.

Many commercial probiotics are available, having various effects of reducting gastrointestinal discomfort or strengthening of the immune system. Preferred probiotic bacteria species for use in the compositions and methods described herein include those commercially available strains of probiotic bacteria (such as sAJ2 bacteria), especially those from the Streptococcus (e.g., S. thermophiles), Bifidobacterium (e.g., B. longum, B. breve, B. infantis, B. breve, B. infantis), and Lactobacillus genera (e.g., L. acidophilus, L. helveticus, L. bulgaricus, L. rhamnosus, L. plantarum, and L. casei). The instant disclosure comprises methods of administering at least one probiotic bacterial strain, preferably a combination of two or more different bacterial strains, to a subject, preferably a mammal (e.g., a human). Such administration may be systemically or locally (e.g., directly to intestines) performed. A preferably administration route is oral administration. Other routes (e.g., rectal) may be also used. For administration, either the bacteria (e.g., in a wet, sonicated, ground, or dried form or formula), the bacterial culture medium containing the bacteria, or the bacterial culture medium supernatant (not containing the bacteria), may be administered.

### Methods of Detection

In certain aspects, provided herein is a method of detecting at least one biomarker. In some embodiments, a biomarker indicates the status of immune function. In some embodiments, the biomarker is the level (secretion level and/or expression level) of a cytokine or chemokine. In some embodiments, the biomarker is the level of IFN-γ. In some embodiments, the biomarker is the activity or activation of NK cells. Such biomarkers are useful in determining whether a subject would benefit from the treatment with certain cannabis of the present disclosure.

In other embodiments, a biomarker is differentially expressed in cancer cells after treatment with cannabis of the present disclosure. Detection of such biomarker(s) allows the identification of the patient pool, determination of the efficacy of the cannabis, and/or prognosis of a subject treated with the cannabis.

Any methods known in the art can be used to determine the level and/or activity of the biomarker. Non-limiting examples of such methods include immunological methods (e.g., ELISA, ELISPOT, immunohistochemistry etc.) for detection of secreted, cell-surface, cytoplasmic, or nuclear proteins, protein purification methods, protein function or activity assays, nucleic acid hybridization methods, nucleic acid reverse transcription methods, and nucleic acid amplification methods.

Exemplary methods in determining the level of a biomarker (e.g., level of IFN-γ or the activity of NK cells) are presented in Examples of the present disclosure. In some embodiments, the NK cell activation is determined by an increase in proliferative capability, cytotoxicity, secretion of at least one cytokine/chemokine, and/or cell surface markers that are associated with activated NK cells (e.g., the level of CD3, CD11b, CD16, CD27, CD56, CD69, CD107a, or a combination thereof). For example, CD3−/CD16+CD56dim phenotype is typical for cytotoxic NK cells, whereas the regulatory CD3-/CD16negCD56bright NK cells mediate differentiation of stem cells in humans. Cell surface markers indicative of NK cell activation are well known in the art, see, e.g., Kozlowska et al. (2016) Cancer Immunol Immunother, 65(7): 835-845; Kozlowska et al. (2017) Front Biosci. 1(22):370-384.

### Predictive Medicine

The present invention provides for methods of determining whether a subject would benefit from the compositions and methods provided herein, as well as the prophylactic and therapeutic methods of treating a subject at risk of (or susceptible to) a cancer. The cancer may be a solid or hematological cancer.

The present invention also pertains to the field of predictive medicine in which diagnostic assays, prognostic assays, and monitoring clinical trials are used for prognostic (predictive) purposes to thereby treat an individual prophylactically. Accordingly, certain aspects encompassed by the present invention relate to diagnostic assays for determining the amount and/or activity level of a biomarker (e.g., secreted level of IFN-γ, activity level of NK cells) described herein in the context of a biological sample (e.g., cancer cells, blood sample, etc.) to thereby determine whether an individual afflicted with a condition that would benefit from a composition comprising cannabis. Such assays can be used for prognostic or predictive purpose alone, or can be coupled with a therapeutic intervention to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those in the figures, examples, and otherwise described in the specification; or one or more biomarkers known in the art (e.g., other cytokines that are secreted by NK cells, cell surface markers that indicates the activity of NK cells).

### Diagnostic Assays

The present invention provides, in part, methods, systems, and code for accurately classifying whether a biological sample is associated with a condition that would benefit from the compositions of the present disclosure. In some embodiments, the present invention is useful for classifying a sample (e.g., from a subject) as associated with or at risk for a condition that would benefit from the cannabis compositions.

An exemplary method for detecting the amount or activity of a biomarker described herein, and thus useful for classifying whether a sample is likely or unlikely to respond to cannabis of the present disclosure involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in series. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely cannabinoid responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method encompassed by the present invention further comprises sending the sample classification results to a clinician, e.g., an oncologist.

In some embodiments, the diagnosis of a subject is followed by administering to the individual a defined treatment based upon the diagnosis.

In some embodiments, the methods further involve obtaining a control biological sample (e.g., biological sample from a subject who does not have a condition that would benefit from cannabis of the present disclosure), a biological sample from the subject during remission, or a biological sample from the subject during treatment for developing a condition that would benefit from cannabis of the present disclosure.

### Prophylactic Methods

In certain aspects, the present invention provides a method for preventing in a subject, a disease or condition associated with cancer. Subjects at risk for a disease that would benefit from treatment with the claimed agents or methods can be identified, for example, by any or a combination of diagnostic or prognostic assays known in the art. Administration of a prophylactic agent can occur prior to the manifestation of symptoms associated with cancer. The appropriate agent used for treatment (e.g. cannabis or cannabis in combination with at least one cancer therapy) can be determined based on clinical indications and can be identified.

### Therapeutic Methods

Another aspect encompassed by the present invention pertains to therapeutic methods of inhibiting the proliferation of a cancer cell by administering the compositions described herein. The therapeutic compositions described herein can be used in a variety of in vitro and in vivo therapeutic applications using the formulations and/or combinations described herein. In some embodiments, the therapeutic agents can be used to treat cancers determined to be responsive thereto. For example, single or combination therapy can be used to treat cancers in subjects identified as likely responders thereto.

Modulatory methods encompassed by the present invention involve contacting a cell, such as a cancer cell, with a composition comprising cannabis described herein. Exemplary compositions useful in such methods are described above. Such compositions can be administered in vitro or ex vivo (e.g., by contacting the cell with the composition) or, alternatively, in vivo (e.g., by administering the agent to a subject). As such, the present invention provides methods useful for treating an individual afflicted with a condition that would benefit from the compositions described herein.

As described above, in certain instances, it may be desirable to further administer an additional therapy, e.g., cancer therapy. In certain embodiments, the method further comprises surgery, radiation therapy, chemotherapy, immunotherapy, or a combination thereof. In certain embodiments, the method further comprises immunotherapy which includes, NK-therapy, CAR-T therapy, and antibody therapy.

In certain embodiments, treatment with a compound or therapy described herein causes the cancer to enter a state of static growth. In some embodiments, said treatment causes cancer cell death. In certain embodiments, the cell death is autophagic, apoptotic, or necrotic.

### Clinical Efficacy

Clinical efficacy can be measured by any method known in the art. For example, the response to a therapy (e.g., cannabis or a combination therapy provided herein), relates to e.g., any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al. (2007) J. Clin. Oncol. 25:4414-4422) or Miller-Payne score (Ogston et al. (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer vaccine therapeutic regimen is at least 25%, 30%, 35%, 40%, 45%, 50%, 55%, 60%, 65%, 70%, 75%, 80%, 85%, or more.

Additional criteria for evaluating the response to a therapy (e.g., cannabis or a combination therapy provided herein) are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular agent encompassed by the present invention can be administered to a population of subjects and the outcome can be correlated to biomarker measurements that were determined prior to administration of a therapy (e.g., cannabis or a combination therapy provided herein). The outcome measurement may be pathologic response to therapy given in the neoadjuvant setting. Alternatively, outcome measures, such as overall survival and disease-free survival can be monitored over a period of time for subjects following a therapy (e.g., cannabis or a combination therapy provided herein). In certain embodiments, the same doses of the agent are administered to each subject. In related embodiments, the doses administered are standard doses known in the art for the agent. The period of time for which subjects are monitored can vary. For example, subjects may be monitored for at least 2, 4, 6, 8, 10, 12, 14, 16, 18, 20, 25, 30, 35, 40, 45, 50, 55, or 60 months.

### EXAMPLES

### Example 1

### Effect of Synthetic Cannabinoids on Well Differentiated and Cancer Stem Cells

### Methods and Materials

### Cell Lines, Reagents, and Antibodies

RPMI 1640 supplemented with 10% Fetal Bovine Serum (FBS) (Gemini Bio-Products, CA) was used for the cultures of immune cells. OSCSCs and OSCCs were dissociated and grown from the tongue tumors of patients at UCLA, and were cultured with RPMI 1640 supplemented with 10% FBS. Recombinant IL-2 was obtained from NIH-BRB. Flow cytometry antibodies used in this study were obtained from Biolegend (San Diego, CA). Monoclonal anti-TNF-α and monoclonal anti-IFN-g antibodies were either obtained from commercial sources or prepared in our laboratory and 1:100 dilution was found to be the optimal concentration to use for blocking experiments as described previously.

### Purification of Human NK Cells and Monocytes

Written informed consents approved by UCLA Institutional Review Board (IRB) were obtained and all procedures were approved by UCLA-IRB. PBMCs from healthy human donors 380 were isolated, and NK cells and monocytes were purified using isolation kits obtained from Stem Cell Technologies, as described before. The purity of NK cells and monocyte populations was found to be 95% or higher, respectively, based on the flow cytometric analysis.

Probiotic Bacteria

AJ2 is a combination of 8 different strains of gram-positive probiotic bacteria (Streptococcus thermophiles, Bifidobacterium longum, Bifidobacterium breve, Bifidobacterium infantis, Lactobacillus acidophilus, Lactobacillus plantarum, Lactobacillus casei, and Lactobacillus bulgaricus) used to induce differentiation of stem cells and are selected for their superior ability to induce optimal secretion of both pro-inflammatory and anti-inflammatory cytokines in NK cells. In addition, each strain was grown, and specific colonies were selected after three rounds of subcloning based on the ability to withstand environmental pressures such as temperature and acidity

### Human Single-Color Enzymatic ELISPOT Assay for IFN-γ

80 μl of anti-human IFN-γ capture antibody was added to each well of a 96-well high-protein-binding PVDF filter plate and incubated overnight at 4° C. The plate was washed with 15011.1 of PBS once before adding samples into the plate. 50,000 cells in 200 μl of RPMI were added into each well and incubate at 37° C., 5% CO2 overnight. After incubation, the plate was washed twice with 200 μl PBS followed by 0.05% 200 μl Tween-PBS twice. 8011.1 of anti-human IFN-γ detection antibody was added into each well and incubated at room temperature for 2 hours and the plate was washed three times with 200 μI/well of 0.05% Tween-PBS. 80 μI/well of tertiary solution which was made from 1:1000 diluted Strep-AP was added in the plate and incubated for 30 minutes. The plate was washed twice with 200 μI/well of 0.05% Tween-PBS followed by 200 μI/well distilled water twice. Then, 80 μI/well of blue development solution was added, and the plate was incubated at room temperature for 15 minutes. The reaction was stopped by gently rinsing membrane with tap water for 3 times. Air-dried the plate for 2 hours and was scanned to count IFN-γ release using CTL machine with immunoSpot® Sofeware. (Cellular Technology Limited, OH, USA).

### ELISA and Multiplex Cytokine Array Kit

Single ELISAs and multiplex assays were performed as described previously. To analyze and obtain the cytokine and chemokine concentration, a standard curve was generated by either two or three-fold dilution of recombinant cytokines provided by the manufacturer. For multiple cytokine array, the levels of cytokines and chemokines were examined by multiplex assay, which was conducted as described in the manufacturer's protocol for each specified kit. Analysis was performed using a Luminex multiplex instrument (MAGPIX, Millipore, Billerica, MA) and data was analyzed using the proprietary software (xPONENT 4.2, Millipore, Billerica, MA).

### Surface Staining Assay

For surface staining, the cells were washed twice using ice-cold PBS+1% BSA (Bovine serum albumin). Predetermined optimal concentrations of specific human monoclonal antibodies were added to 1×10⁴ cells in 50 μl of cold PBS+1% BSA and cells were incubated on ice for 30 min. Thereafter cells were washed in cold PBS+1% BSA and brought to 500 μl with PBS+1% BSA. Flow cytometric analysis was performed using Beckman Coulter Epics XL cytometer (Brea, CA) and results were analyzed in FlowJo vX software (Ashland, OR).

### ⁵¹Cr Release Cytotoxicity Assay

The ⁵¹Cr release assay was performed as described previously. Briefly, different numbers of effector cells were incubated with ⁵¹Cr-labeled target cells. After a 4-hour incubation period the supernatants were harvested from each sample and counted for released radioactivity using the gamma counter. The percentage specific cytotoxicity was calculated as follows:

$$\% Cytotoxicity = \frac{Experimental cpm - spontaneous cpm}{Total cpm - spontaneous cpm}$$

LU 30/106 is calculated by using the inverse of the number of effector cells needed to lyse 30% of tumor target cells×100.

### Target Cell Visualization Assay (TVA)

Target cells were incubated with TVATM dye at 370 C for 15 mins, afterwards effector cells were cultured with target cells for 4 hours. After a 4-hour incubation period the target cells were counted with immunospot at 525 nm emission wavelengths. The percentage specific cytotoxicity was calculated as follows:

$$\% Cytotoxicity = \frac{Experimental cpm - spontaneous cpm}{Total cpm - spontaneous cpm}$$

LU 30/107 is calculated by using the inverse of the number of effector cells needed to lyse 30% of tumor target cells×100.

Statistical Analysis

The prism-7 software is used for the statistical analysis. An unpaired or paired, two-tailed student t-test was performed for the statistical analysis. One-way ANOVA with a Bonferroni post-test was used to compare different groups. (n) denotes the number of human donors or mice. For in-vitro studies either duplicate or triplicate samples were used for assessment. The following symbols represent the levels of statistical significance within each analysis, ***(p value <0.001), **(p value 0.001-0.01), *(p value 0.01-0.05).

Cell Death Assays

Oral Squamous Carcinoma Cells (OSCCs) and stem-like Oral Squamous Carcinoma Stem Cells (OSCSCs) were isolated from tumors of oral cancer patients at UCLA, and cultured in RPMI 1640 supplemented with 10% FBS (Gemini Bio-Products, CA, USA), 1.4% antibiotic antimycotic, 1% sodium pyruvate, 1.4% non-essential amino acids, 1% L-glutamine, 0.2% gentamicin (Gemini Bio-Products, CA, USA), and 0.15% sodium bicarbonate (Fisher Scientific, PA, USA).

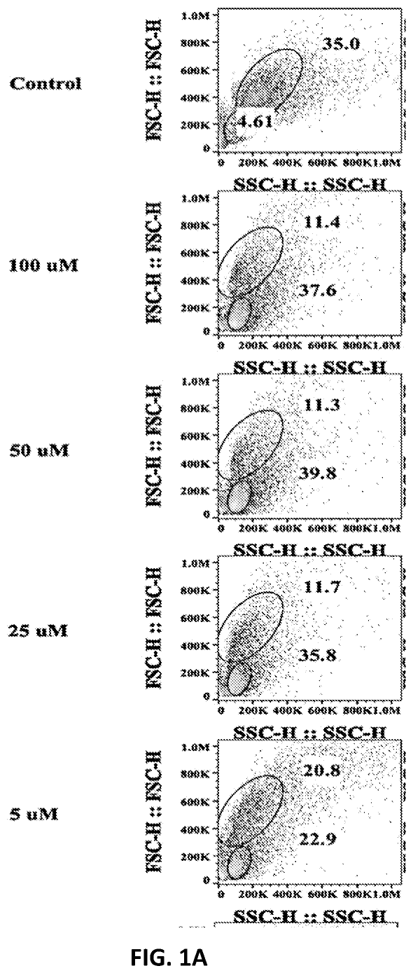

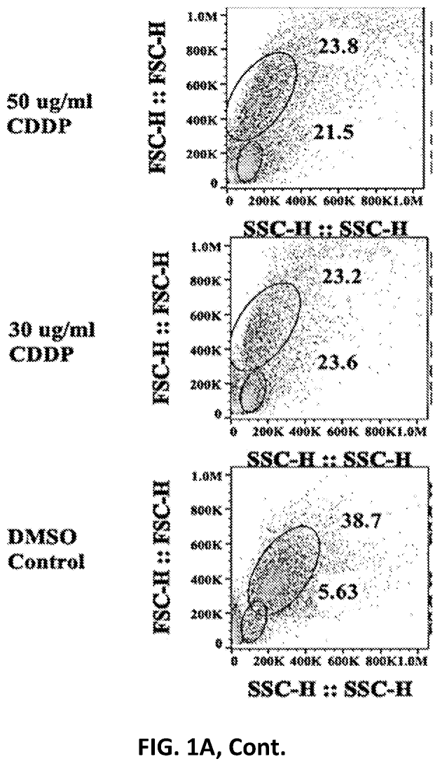

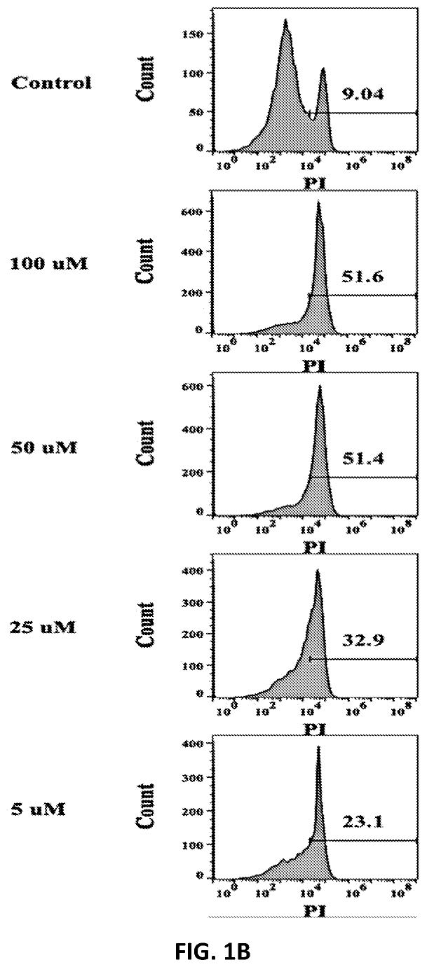

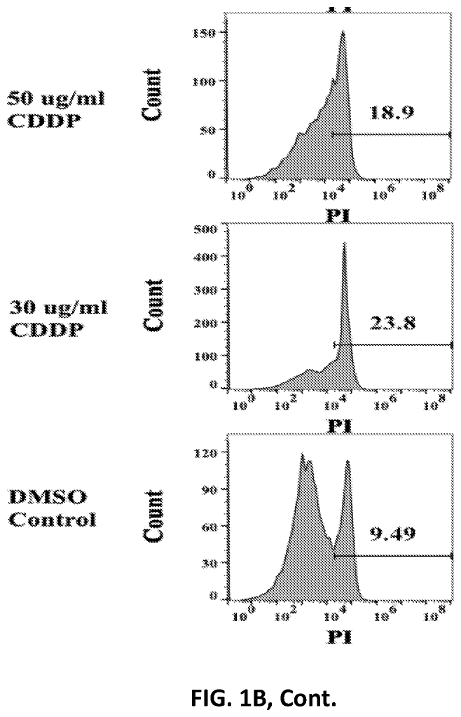

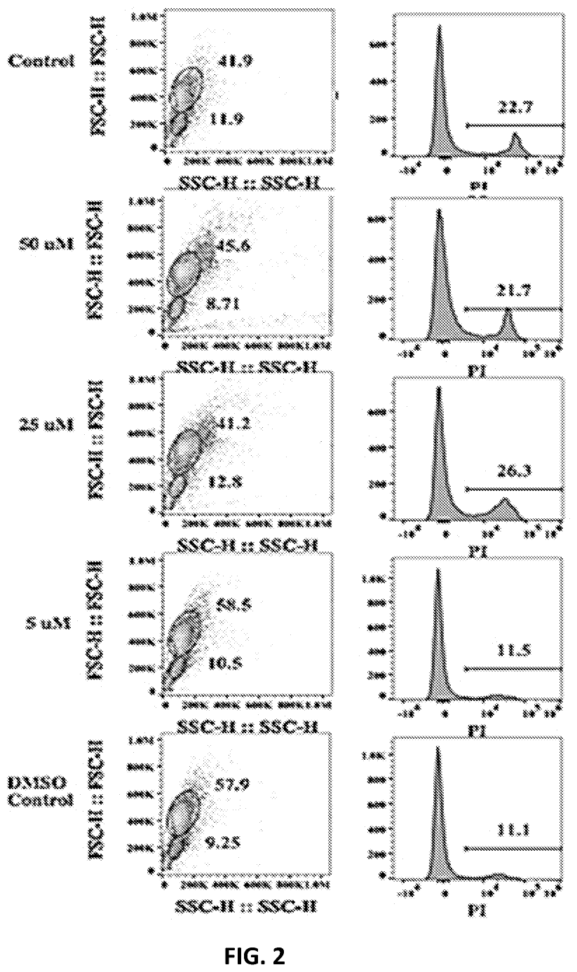

OSCCs and OSCSCs were treated with different concentrations of Win55,2122-2 and cis-diamminedichloridoplatinum (II) (CDDP) as shown in FIGS. 1A-1B, FIG. 2 and Tables 1 and 2, and cultured overnight at 37 C in 5% CO2.

After an overnight incubation the cells were washed twice and stained with Propidium iodide at a concentration of 3 mg/ml, as described by Jewett, A., et al., J Immunol, 1997. 159(10): p. 4815-22; Jewett, A. and B. Bonavida, J Clin Immunol, 1995. 15(1): p. 35-44; and Jewett, A. and B. Bonavida, J Immunol, 1996. 156(3): p. 907-15, each of which is incorporated herein by reference in its entirety.

Flow cytometric analysis was performed using Attune flow cytometer. Briefly, cells were analyzed based on Forward angle light scatter (FS) and Side scatter (SS), and the proportions of cells that had lost FS and became small were determined in total populations of tumor cells. Untreated and DMSO treated tumors were used as controls. In addition, the proportions of cells that were stained with PI were also determined.

OSCCs are differentiated oral squamous carcinoma cells; OSCCs were treated with different concentrations of Win55,2122-2, as indicated in FIGS. 1A-1B and Table 1. FIG. 1A shows the levels of decrease in Forward Scatter in the cells representing loss of cell morphology closely associated with the induction of cell death (FIG. 1A and Table 1), as well as cell death, which was determined using Propidium Iodide (PI) staining (FIG. 1B and Table 1). As can be seen in FIG. 1A, Win55,2122-2 decreased FS even at the lower concentrations of drug, and a plateauing effect was seen until 5 μM levels (FIG. 1A and Table 1). When cell death was determined by staining with PI significant cell death was also observed at higher concentrations and titration could be seen at 25 and 5 μM of Win55,2122-2. Therefore, Win55,2122-2 is a potent inducer of cell death in differentiated oral tumors.

TABLE 1
Levels of forward scatter and cell death in Oral Squamous Carcinoma
Cells (OSCCs) treated with different concentrations of Win55,2122-2.
OSCC

| % decrease in FS | | | | % PI | | |
|---|---|---|---|---|---|---|
| | | | | | | Percent cell death |
| | | | Percent of cells | | | induced in total |
| Concentration | | | that lost FS in | Concentration | | population of |
| of | | Ratio | total population | of | | tumor cells, as |
| Win55,2122-2 | | represents | of tumor cells | Win55,2122-2 | | visualized by PI |
| Control | | 35.0/4.61 | 9% | Control | | 9.04% |
| 100 | μM | 11.4/37.6 | 77% | 100 | μM | 51.6% |
| 50 | μM | 11.3/39.8 | 78% | 50 | μM | 51.6% |
| 25 | μM | 11.7/35.8 | 75% | 25 | μM | 32.9% |
| 5 | μM | 20.8/22.9 | 52% | 5 | μM | 23.1% |

| | | | | |
|---|---|---|---|---|
| 50 μg/ml CDDP | 23.8/21.5 | 53% | 50 μg/ml CDDP | 18.9% |
| 30 μg/ml CDDP | 23.2/23.6 | 51% | 30 μg/ml CDDP | 23.8% |
| DMSO | 38.7/5.63 | 11% | DMSO | 9.49% |

OSCSCs are stem like oral squamous carcinoma cells; OSCSCs were treated with different concentrations of Win55,2122-2, as indicated in FIG. 2 and Table 2. FIG. 2 shows the levels of decrease in Forward Scatter in the cells and cell death induced by Win55,2122-2, determined using Propidium Iodide (PI) staining. As can be seen in FIG. 2, Win55,2122-2 did not change FS even at the higher concentrations of drug. When cell death was determined by staining with PI, no or moderate levels of cell death was observed even at higher concentrations of Win55,2122-2 when compared to untreated or DMSO treated tumors. Therefore, Win55,2122-2 does not induce significant cell death in OSCSCs unlike in OSCCs.

TABLE 2
Levels of forward scatter and cell death in Oral
Squamous Carcinoma Stem Cells (OSCSCs) treated
with different concentrations of Win55,2122-2.
OSCSCs

| % of decrease in FS | | | | % PI | | |
|---|---|---|---|---|---|---|
| | | | Percent of | | | Percent of |
| | | | cells that lost | | | cells that lost |
| Concentration | | | FS in total | Concentration | | FS in total |
| of | | Ratio | population of | of | | population of |
| Win55,2122-2 | | represents | tumor cells | Win55,2122-2 | | tumor cells |
| Control | | 41.9/11.9 | 22% | Control | | 22.7% |
| 50 | μM | 45.6/8.71 | 16% | 50 | μM | 21.7% |
| 25 | μM | 41.2/12.8 | 24% | 25 | μM | 26.3% |
| 5 | μM | 58.5/10.5 | 15% | 5 | μM | 11.5% |

| | | | | |
|---|---|---|---|---|
| DMSO | 57.9/9.25 | 14% | DMSO | 11.1% |

### Example 2

### Preparation of Therapeutic Cannabis Oil

Raw, organic cannabis flower undergoes a CO2 extraction process that creates crude oil. This is followed by an ethanol winterization process, which turns the crude oil into distillate, thus leaving the plant material and stripping away most of the terpenes. Then, the distillate oil is diluted with organic medium-chain triglyceride (MCT) oil, followed by an infusion of a specific blend of plant-based terpenoids that results in the finished product.

### Example 3

### Higher Secretion or Restoration of IFN-γ Secretion in NK and PBMCs of Cancer Patients after Ingestion of Cannabis Compared to Healthy Controls and Patients not Ingesting Cannabis

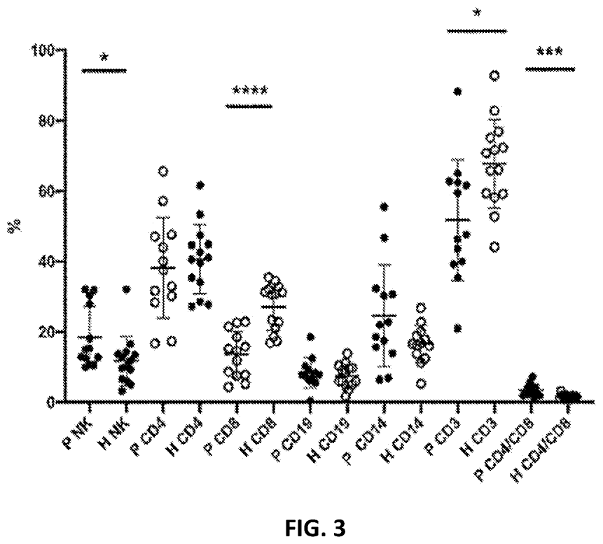

Flow cytometry was performed to determine the proportion of NK, CD3, CD4, CD8, CD19, and CD14 cells as well as the ratio of CD4/CD8 in PBMC in healthy donors and cancer patients after cannabis ingestion. Percent NK cells and CD4/CD8 ratio in patient's PBMC is significantly higher than healthy donor's, whereas patient PBMC contains lower percentage of total CD3+ T cells as well as lower CD8+ T cell than healthy donor's, as shown in FIG. 3 and Table 3.

Increased NK cell cytotoxicity was demonstrated by NK cells from patients who ingested cannabis (“on cannabis”) as compared to healthy controls when compared to data from patients who did not ingest cannabis.

TABLE 3
Flow cytometry results to determine the proportion of NK, CD3, CD4, CD8,
CD19, and CD14 cells, as well as the ratio of CD4/CD8 in PBMC in Cannabis
patients and healthy donors (control). MEAN + SD or SE (based on FIG. 3).

| | NK | | CD3 | | CD4 | | CD8 | | CD4/CD8 | | CD19 | | CD14 | |
|---|---|---|---|---|---|---|---|---|---|---|---|---|---|---|
| | Mean | SD | Mean | SD | Mean | SD | Mean | SD | Mean | SD | Mean | SD | Mean | SD |
| Patient on | 17.7 | 8.6 | 52.8 | 17.6 | 39.0 | 14.6 | 13.5 | 6.8 | 3.6 | 1.6 | 8.7 | 4.4 | 24.0 | 15.0 |
| Cannabis | | | | | | | | | | | | | | |
| Healthy | 11.8 | 7.0 | 67.7 | 12.6 | 40.6 | 9.8 | 27.1 | 6.7 | 1.6 | 0.6 | 7.5 | 3.3 | 16.5 | 5.4 |

| | NK | | CD3 | | CD4 | | CD8 | | CD4/CD8 | | CD19 | | CD14 | |
|---|---|---|---|---|---|---|---|---|---|---|---|---|---|---|
| | Mean | SE | Mean | SE | Mean | SE | Mean | SE | Mean | SE | Mean | SE | Mean | SE |
| Patient on | 17.7 | 2.5 | 52.8 | 5.1 | 39.0 | 4.2 | 13.5 | 2.0 | 3.6 | 0.5 | 8.7 | 1.3 | 24.0 | 4.3 |
| Cannabis | | | | | | | | | | | | | | |
| Healthy | 11.8 | 1.9 | 67.7 | 3.4 | 40.6 | 2.6 | 27.1 | 1.8 | 1.6 | 0.2 | 7.5 | 0.9 | 16.5 | 1.5 |

### Example 4

Increased NK cell cytotoxicity from patients on cannabis as compared to healthy controls when compared to data from patients without the use of cannabis PBMCs (1×10⁶ cells/ml) from healthy donors and cancer patients with and without Cannabis use were left untreated or treated with IL-2 (1000 U/ml), or the combination of IL-2 and anti-CD16mAb (3 μg/ml) or IL-2 and combination of anti-CD3/CD28 antibodies, and IL-2 in combination with sAJ2 (20:1, bacteria:PBMCs) for 18 hours before they were added to ⁵¹Cr labeled oral squamous cell carcinoma stem cells (OSCSCs) at various effector to target ratios.

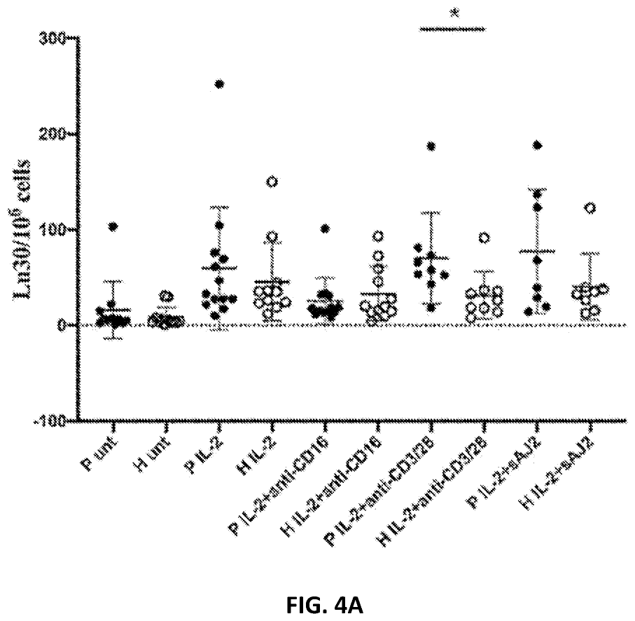

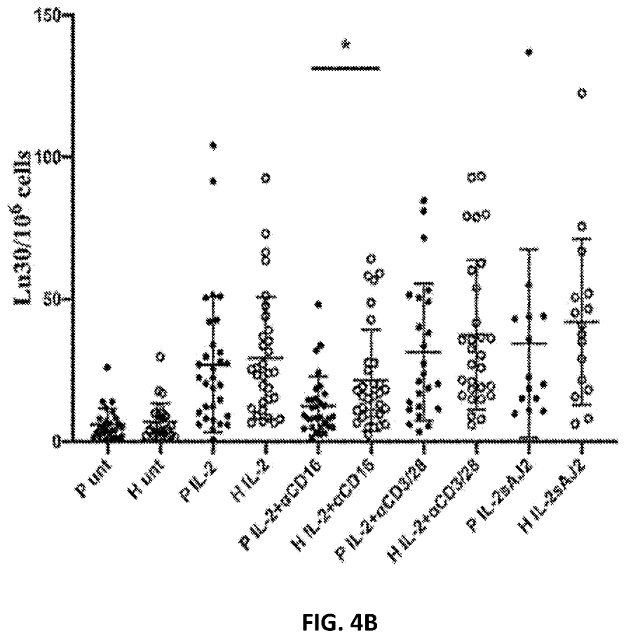

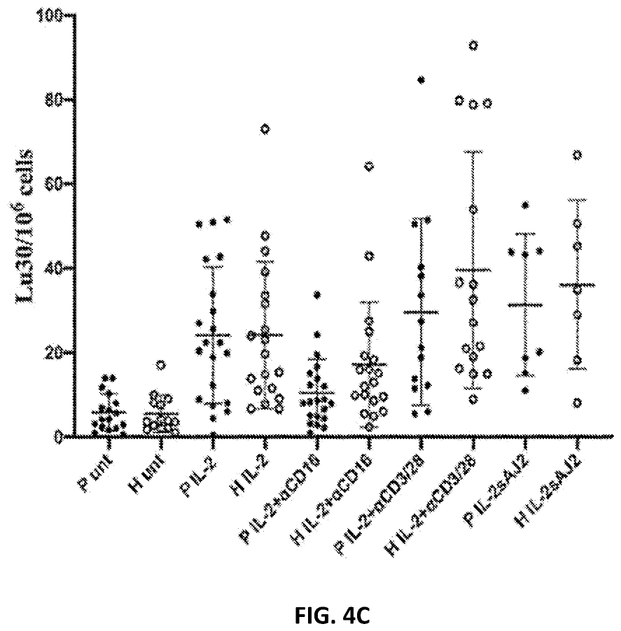

NK cell-mediated cytotoxicity using a standard 4-hour ⁵¹Cr release assay against the oral squamous cell carcinoma stem cell line (OSCSCs) were performed and the lytic units 30/10⁶ cells were determined using the inverse number of lymphocytes required to lyse 30% of OSCSCs×100. Chromium release assay was performed with PBMC obtained from Cannabis Patients (FIG. 4A), All Cancer Patients (FIG. 4B), Pancreatic Cancer Patients (FIG. 4C).

NK cells from patients demonstrated increased cytotoxicity when compared to NK celled from healthy controls and when compared to data from patients who did not ingest cannabis.

TABLE 4
Chromium Release Assay PBMC (Mean and SD
or SEM of patients based on FIGS. 4A-4C.)

| | | | | | IL-2 + anti- | | IL-2 + anti- | | | |
|---|---|---|---|---|---|---|---|---|---|---|
| | untreated | | IL-2 | | CD16 | | CD3/28 | | IL-2 + sAJ2 | |
| | Mean | SD | Mean | SD | Mean | SD | Mean | SD | Mean | SD |
| Patient on | 16.0 | 29.6 | 59.5 | 63.9 | 25.3 | 24.2 | 70.1 | 47.5 | 77.1 | 64.6 |
| Cannabis | | | | | | | | | | |
| Healthy | 8.6 | 10.2 | 45.3 | 40.8 | 32.9 | 28.3 | 31.2 | 24.7 | 40.3 | 34.7 |
| donor | | | | | | | | | | |
| All cancer | 6.02 | 5.60 | 26.99 | 23.85 | 12.47 | 10.37 | 31.43 | 24.10 | 34.44 | 33.11 |
| patient | | | | | | | | | | |
| Healthy | 6.93 | 6.49 | 29.34 | 21.42 | 21.45 | 17.83 | 37.48 | 26.30 | 42.00 | 29.21 |
| donor | | | | | | | | | | |
| Pancreatic | 5.80 | 4.41 | 24.07 | 16.17 | 10.42 | 8.03 | 29.65 | 22.12 | 31.36 | 16.81 |
| cancer | | | | | | | | | | |
| patient | | | | | | | | | | |
| Healthy | 5.45 | 4.42 | 24.10 | 17.40 | 17.15 | 14.84 | 39.60 | 28.05 | 36.14 | 20.03 |
| donor | | | | | | | | | | |
| | | | | | IL-2 + anti- | | IL-2 + anti- | | | |
| | untreated | | IL-2 | | CD16 | | CD3/28 | | IL-2 + sAJ2 | |
| | Mean | SE | Mean | SE | Mean | SE | Mean | SE | Mean | SE |
| Patient on | 16.0 | 8.9 | 59.5 | 17.7 | 25.3 | 6.7 | 70.1 | 15.8 | 77.1 | 22.9 |
| Cannabis | | | | | | | | | | |
| Healthy | 8.6 | 2.9 | 45.3 | 12.3 | 32.9 | 8.2 | 31.2 | 8.2 | 40.3 | 12.3 |
| donor | | | | | | | | | | |
| All cancer | 6.02 | 1.10 | 26.99 | 4.28 | 12.47 | 1.86 | 31.43 | 4.92 | 34.44 | 8.85 |
| patient | | | | | | | | | | |
| Healthy | 6.93 | 1.27 | 29.34 | 3.79 | 21.45 | 3.15 | 37.48 | 4.88 | 42.00 | 7.30 |
| donor | | | | | | | | | | |
| Pancreatic | 5.80 | 1.07 | 24.07 | 3.53 | 10.42 | 1.75 | 29.65 | 5.91 | 31.36 | 5.94 |
| cancer | | | | | | | | | | |
| patient | | | | | | | | | | |
| Healthy | 5.45 | 1.18 | 24.10 | 3.99 | 17.15 | 3.41 | 39.60 | 7.01 | 36.14 | 7.57 |
| donor | | | | | | | | | | |

### Example 5

### Increased IFN-γ Spots in PBMCs from Patients on Cannabis as Compared to Healthy Controls when Compared to Data from Patients without the Use of Cannabis

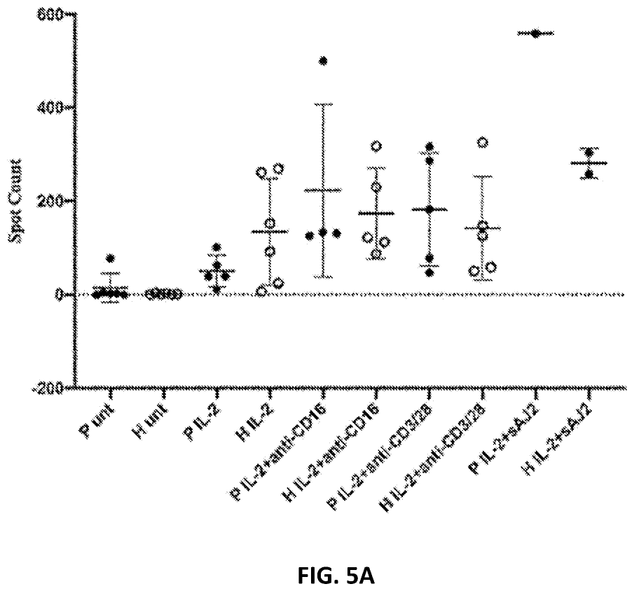

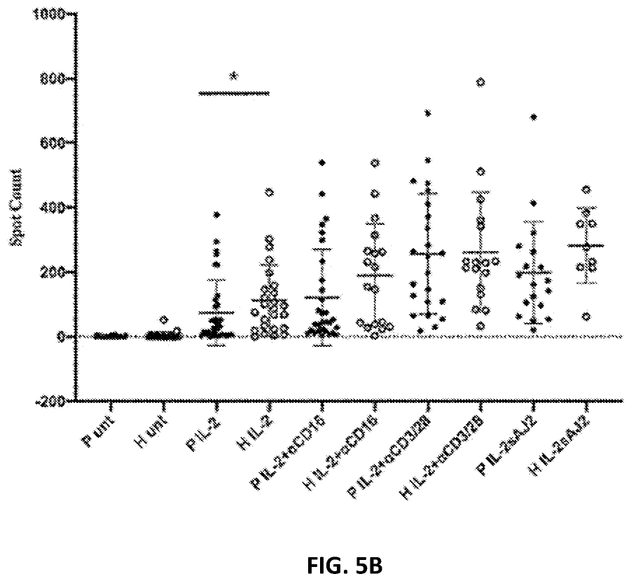

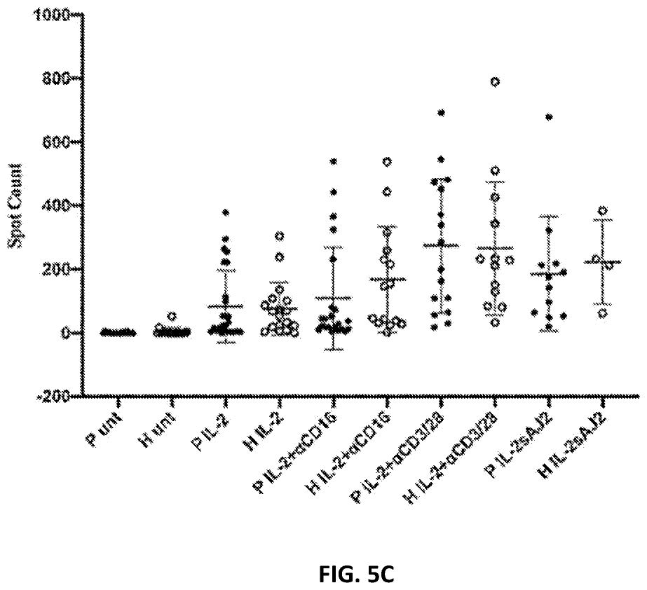

PBMCs were isolated as described in the M&M section and an enzyme-linked immunospot (ELISPOT) assay was conducted to determine the numbers of IFN-γ spots representing the number of cells expressing IFN-γ. PBMCs were treated with IL-2 (1000 U/ml) or the combination of IL-2 and anti-CD16mAb (3 □g/ml) or IL-2 and combination of anti-CD3/CD28 antibodies or IL-2 in combination with sAJ2. The number of IFN-γ spots was higher in patient's PBMCs on Cannabis when compared to healthy controls. Specifically, treatment with IL-2 and anti-CD16mAb, IL-2+anti-CD3/CD28 antibodies and IL-2+sAJ2. When compared to the sets of patients and healthy without the use of Cannabis the profiles were reversed, with healthy having higher increase in the spots than patients. ELISPOT was performed on PBMCs from Cannabis Patients (FIG. 5A), All Cancer Patients (FIG. 5B), Pancreatic Cancer Patients (FIG. 5C).

ELISPOT of PBMCs from patients who ingested cannabis (“on cannabis”) demonstrated increased IFN-γ spots as compared to healthy controls when compared to data from patients who did not ingest cannabis, as shown in FIGS. 5A-5C and Table 5.

TABLE 5
ELISPOT for IFN-γ spots in PBMCs of patients ingested cannabis, healthy subjects
and patients who did not ingest cannabis (mean with SD or SEM) based on FIGS. 5A-5C.

| | | | | | IL-2 + anti- | | IL-2 + anti- | | | |
|---|---|---|---|---|---|---|---|---|---|---|
| | untreated | | IL-2 | | CD16 | | CD3/28 | | IL-2 + sAJ2 | |
| | Mean | SD | Mean | SD | Mean | SD | Mean | SD | Mean | SD |
| Patient on | 14.6 | 31.1 | 50.4 | 33.5 | 222.5 | 185.0 | 181.8 | 120.0 | 559.0 | 120.0 |
| Cannabis | | | | | | | | | | |
| Healthy | 1.6 | 1.6 | 134.2 | 113.7 | 173.6 | 97.1 | 141.4 | 110.8 | 280.5 | 110.8 |
| donor | | | | | | | | | | |
| All cancer | 1.02 | 1.81 | 73.96 | 101.50 | 121.00 | 149.10 | 256.70 | 186.10 | 198.40 | 158.40 |
| patient | | | | | | | | | | |
| Healthy | 4.05 | 11.63 | 112.10 | 109.80 | 189.40 | 159.50 | 262.00 | 184.10 | 281.80 | 116.90 |
| donor | | | | | | | | | | |
| Pancreatic | 0.98 | 1.68 | 83.17 | 112.50 | 108.80 | 160.40 | 274.00 | 211.10 | 185.30 | 178.90 |
| cancer | | | | | | | | | | |
| patient | | | | | | | | | | |
| Healthy | 4.53 | 12.90 | 75.50 | 82.09 | 167.70 | 165.40 | 265.50 | 209.20 | 222.50 | 131.30 |
| donor | | | | | | | | | | |

| | | | | | IL-2 + anti- | | IL-2 + anti- | | | |
|---|---|---|---|---|---|---|---|---|---|---|
| | untreated | | IL-2 | | CD16 | | CD3/28 | | IL-2 + sAJ2 | |
| | Mean | SE | Mean | SE | Mean | SE | Mean | SE | Mean | SE |
| Patient on | 14.6 | 12.7 | 50.4 | 15.0 | 222.5 | 92.5 | 181.8 | 53.7 | 559.0 | 0.0 |
| Cannabis | | | | | | | | | | |
| Healthy | 1.6 | 0.7 | 134.2 | 46.4 | 173.6 | 43.4 | 141.4 | 49.6 | 280.5 | 22.5 |
| donor | | | | | | | | | | |
| All cancer | 1.02 | 0.34 | 73.96 | 17.40 | 121.00 | 27.22 | 256.70 | 38.81 | 198.40 | 37.33 |
| patient | | | | | | | | | | |
| Healthy | 4.05 | 2.54 | 112.10 | 21.96 | 189.40 | 37.60 | 262.00 | 44.66 | 281.80 | 38.98 |
| donor | | | | | | | | | | |
| Pancreatic | 0.98 | 0.35 | 83.17 | 22.07 | 108.80 | 34.19 | 274.00 | 52.77 | 185.30 | 51.65 |
| cancer | | | | | | | | | | |
| patient | | | | | | | | | | |
| Healthy | 4.53 | 3.13 | 75.50 | 19.35 | 167.70 | 42.69 | 265.50 | 58.01 | 222.50 | 65.64 |
| donor | | | | | | | | | | |

### Example 6

### Increased IFN-γ Release from PBMCs of Patients on Cannabis as Compared to Healthy Controls when Compared to Data from Patients not on Cannabis

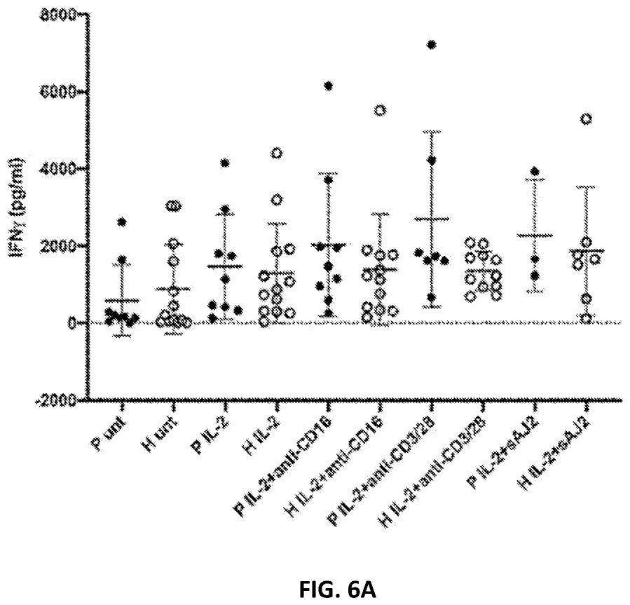

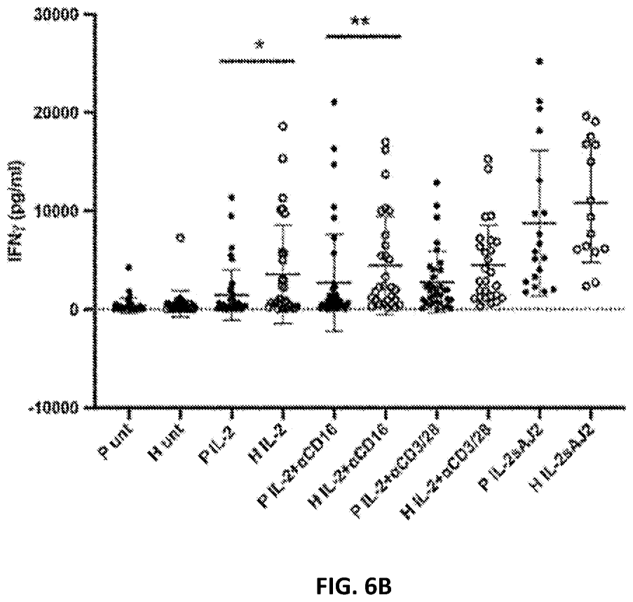

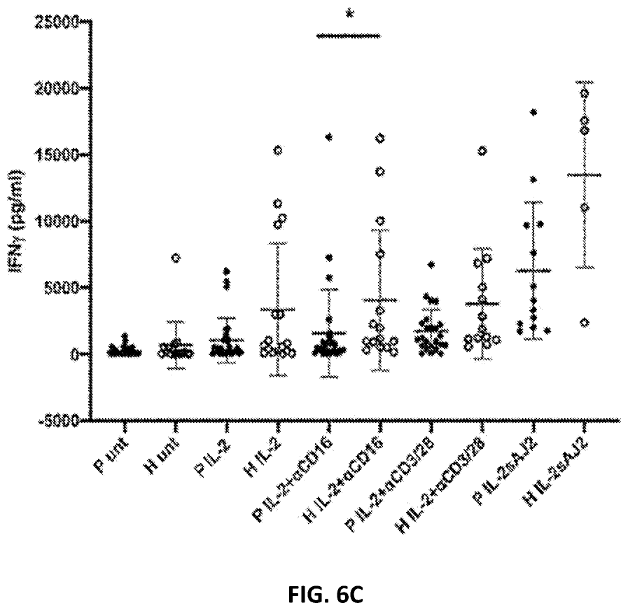

PBMCs were isolated as described in the Materials & Methods (Example 1) section and ELISA was conducted to determine the amount of IFN-γ secretion in the supernatants. PBMCs were treated with IL-2 (1000 U/ml) or the combination of IL-2 and anti-CD16mAb (3 μg/ml) or IL-2 and combination of anti-CD3/CD28 antibodies or IL-2 in combination with sAJ2. The levels of IFN-γ secretion were higher in patient's PBMCs on Cannabis when compared to healthy controls. Specifically, treatment with IL-2 and anti-CD16mAb, IL-2+anti-CD3/CD28 antibodies and IL-2 with sAJ2. When compared to the sets of patients and healthy without the use of Cannabis the profiles were reversed, with healthy having higher increase in the spots than patients. ELISA was performed with supernatant from PBMC Cannabis Patient (FIG. 6A), All Cancer Patients (FIG. 6B), Pancreatic Cancer Patients (FIG. 6C).

ELISA demonstrated increased IFN-γ release from PBMCs of patients on Cannabis as compared to healthy controls when compared to data from patients without the use of Cannabis, as shown in FIGS. 6A-6C and Table 6.

TABLE 6
ELISA for IFN-γ spots in PBMCs of patients ingested cannabis, healthy subjects
and patients who did not ingest cannabis (mean with SD or SEM) based on FIGS. 6A-6C.

| | | | | | IL-2 + anti- | | IL-2 + anti- | | | |
|---|---|---|---|---|---|---|---|---|---|---|
| | untreated | | IL-2 | | CD16 | | CD3/28 | | IL-2 + sAJ2 | |
| | Mean | SD | Mean | SD | Mean | SD | Mean | SD | Mean | SD |
| Patient on | 583.1 | 912.7 | 1458.0 | 1361.0 | 2023.0 | 1844.0 | 2697.0 | 2269.0 | 2266.0 | 1452.0 |
| Cannabis | | | | | | | | | | |
| Healthy | 883.3 | 1158.0 | 1293.0 | 1279.0 | 1385.0 | 1440.0 | 1352.0 | 508.3 | 1867.0 | 1664.0 |
| donor | | | | | | | | | | |
| All cancer | 382.5 | 776.3 | 1457.0 | 2570.0 | 2710.0 | 4931.0 | 2760.0 | 3104.0 | 8740.0 | 7398.0 |
| patient | | | | | | | | | | |
| Healthy | 543.0 | 1326.0 | 3559.0 | 4984.0 | 4457.0 | 4986.0 | 4500.0 | 4035.0 | 10831.0 | 6078.0 |
| donor | | | | | | | | | | |
| Pancreatic | 217.6 | 326.2 | 1054.0 | 1670.0 | 1576.0 | 3277.0 | 1729.0 | 1641.0 | 6270.0 | 5141.0 |
| cancer | | | | | | | | | | |
| patient | | | | | | | | | | |
| Healthy | 680.9 | 1773.0 | 3347.0 | 4960.0 | 4043.0 | 5270.0 | 3794.0 | 4143.0 | 13481.0 | 6975.0 |
| donor | | | | | | | | | | |

| | | | | |
|---|---|---|---|---|
| | | IL-2 + anti- | IL-2 + anti- | |
| untreated | IL-2 | CD16 | CD3/28 | IL-2 + sAJ2 |

| | Mean | SE | Mean | SE | Mean | SE | Mean | SE | Mean | SE |
|---|---|---|---|---|---|---|---|---|---|---|
| Patient on | 583.1 | 304.2 | 1458.0 | 453.6 | 2023.0 | 614.7 | 2697.0 | 857.5 | 2266.0 | 838.3 |
| Cannabis | | | | | | | | | | |
| Healthy | 883.3 | 321.2 | 1293.0 | 354.8 | 1385.0 | 415.6 | 1352.0 | 153.3 | 1867.0 | 629.1 |
| donor | | | | | | | | | | |
| All cancer | 382.5 | 127.6 | 1457.0 | 406.4 | 2710.0 | 779.7 | 2760.0 | 532.3 | 8740.0 | 1697.0 |
| patient | | | | | | | | | | |
| Healthy | 543.0 | 246.2 | 3559.0 | 895.2 | 4457.0 | 925.8 | 4500.0 | 776.5 | 10831.0 | 1569.0 |
| donor | | | | | | | | | | |
| Pancreatic | 217.6 | 82.8 | 1054.0 | 310.0 | 1576.0 | 608.5 | 1729.0 | 335.0 | 6270.0 | 1426.0 |
| cancer | | | | | | | | | | |
| patient | | | | | | | | | | |
| Healthy | 680.9 | 443.1 | 3347.0 | 1203.0 | 4043.0 | 1361.0 | 3794.0 | 1149.0 | 13481.0 | 3119.0 |
| donor | | | | | | | | | | |

### Example 7

### Increased NK Cell Cytotoxicity from Patients Ingesting Cannabis as Compared to Healthy Controls when Compared to Data from Patients not Ingesting Cannabis

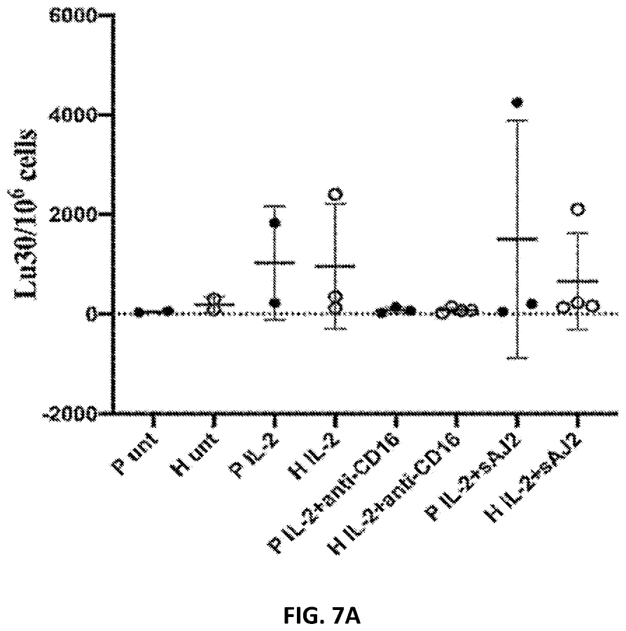

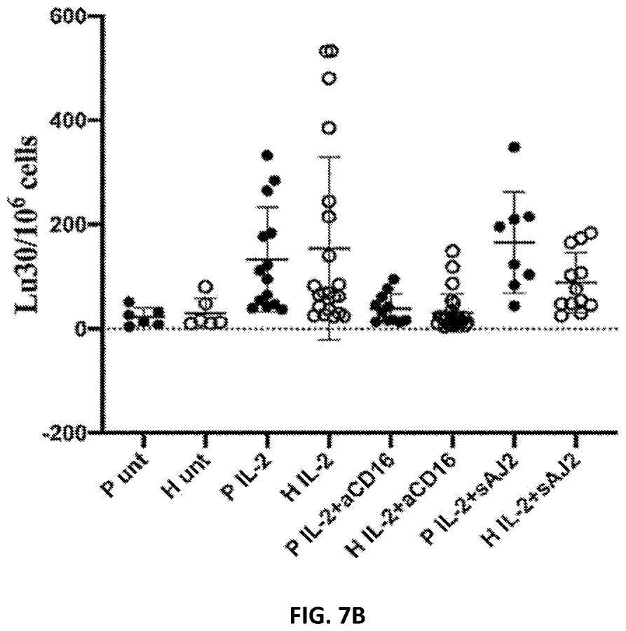

Purified NK cells (1×10⁶ cells/nil) from healthy donors and cancer patients with and without Cannabis use were left untreated or treated with IL-2 (1000 U/ml), or the combination of IL-2 and anti-CD16mAb (3 μg/ml) or IL-2 in combination with sAJ2 (20:1 bacteria: PBMCs) for 18 hours before they were added to 51 Cr labeled oral squamous cell carcinoma stem cells (OSCSCs) at various effector to target ratios. NK cell-mediated cytotoxicity using a standard 4-hour 51 Cr release assay against the oral squamous cell carcinoma stem cell line (OSCSCs) were performed and the lytic units 30/10⁶ cells were determined using the inverse number of lymphocytes required to lyse 30% of OSCSCs×100. Chromium release assay was performed with purified NK cells obtained from Cannabis Patients (FIG. 7A), All Cancer Patients (FIG. 7B).

NK cells from patients who ingested cannabis demonstrated increased cytotoxicity when compared to NK cells from healthy controls and when compared to patients who did not ingest cannabis.

TABLE 7
Chromium_NK (Mean and SD or SEM)

| | | | | | IL-2 + anti- | | | |
|---|---|---|---|---|---|---|---|---|
| | untreated | | IL-2 | | CD16 | | IL-2 + sAJ2 | |
| | Mean | SD | Mean | SD | Mean | SD | Mean | SD |
| Patient on | 40.7 | 19.4 | 1023.0 | 1138.0 | 77.1 | 63.0 | 1501.0 | 2384.0 |
| Cannabis | | | | | | | | |
| Healthy | 190.9 | 156.0 | 956.2 | 1257.0 | 76.0 | 51.1 | 655.3 | 963.9 |
| donor | | | | | | | | |
| All cancer | 22.67 | 17.65 | 132.8 | 100.2 | 38.99 | 28.33 | 165.5 | 96.76 |
| patient | | | | | | | | |
| Healthy | 29.94 | 28.88 | 154.1 | 175.5 | 30.86 | 36.3 | 88.6 | 57.73 |
| donor | | | | | | | | |

| | | | | | IL-2 + anti- | | | |
|---|---|---|---|---|---|---|---|---|
| | untreated | | IL-2 | | CD16 | | IL-2 + sAJ2 | |
| | Mean | SE | Mean | SE | Mean | SE | Mean | SE |
| Patient on | 40.7 | 13.7 | 1023.0 | 804.4 | 77.1 | 36.4 | 1501.0 | 1376.0 |
| Cannabis | | | | | | | | |
| Healthy | 190.9 | 110.3 | 956.2 | 725.8 | 76.0 | 25.5 | 655.3 | 482.0 |
| donor | | | | | | | | |
| All cancer | 22.67 | 7.204 | 132.8 | 26.78 | 38.99 | 8.541 | 165.5 | 34.21 |
| patient | | | | | | | | |
| Healthy | 29.94 | 11.79 | 154.1 | 38.29 | 30.86 | 7.261 | 88.6 | 16.66 |
| donor | | | | | | | | |

### Example 8

### Increased IFN-γ Spots from the NK Cells from Patients on Cannabis as Compared to Healthy Controls when Compared to Data from Patients without the Use of Cannabis

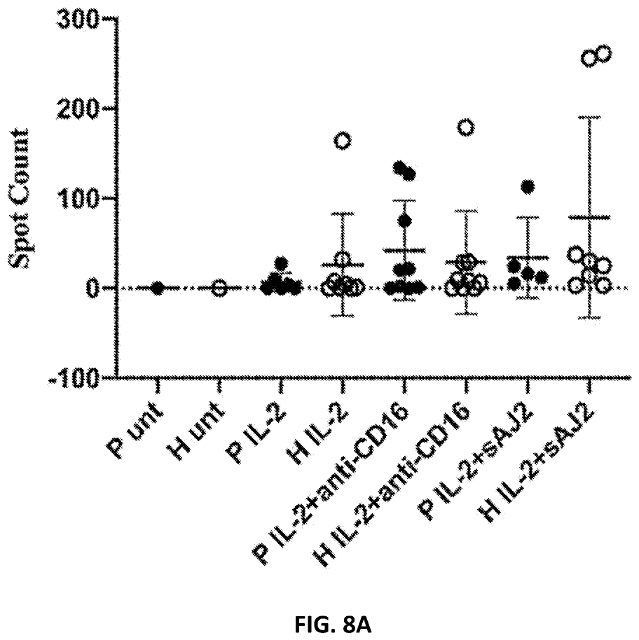

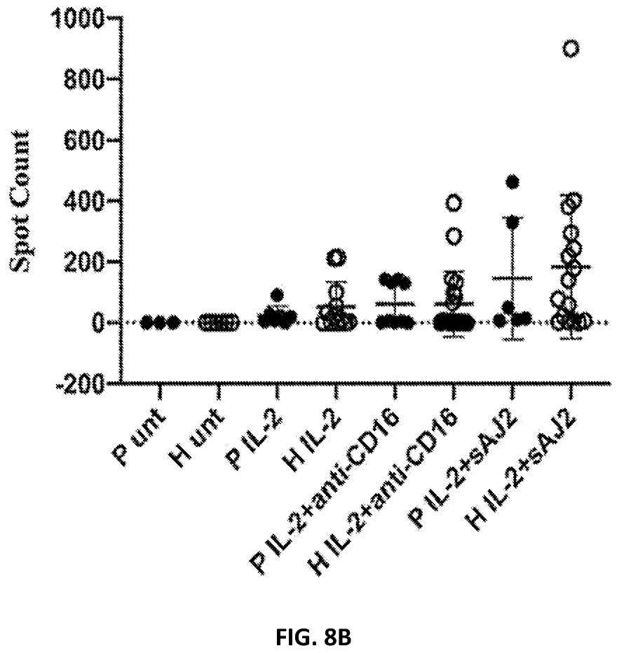

Purified NK cells were isolated as described in the M&M section and Elispot was conducted to determine the numbers of IFN-γ spots representing the number of cells expressing IFN-γ. NK cells were treated with IL-2 (1000 U/ml) or the combination of IL-2 and anti-CD16mAb (3 μg/ml) or IL-2 in combination with sAJ2. The number of IFN-γ spots was variable in patient's NKs on Cannabis when compared to healthy controls. ELISPOT was performed on purified NK cells from Cannabis Patients (FIG. 8A), All Cancer Patients (FIG. 8B).

NK cells from patients on cannabis demonstrated increased IFN-γ spots when compared to healthy controls and when compared to data from patients who did not ingest cannabis.

TABLE 8
ELISPOT_NK (MEAN with SD or SEM) based on FIG. 8A-8B.

| | | | | | IL-2 + anti- | | | |
|---|---|---|---|---|---|---|---|---|
| | untreated | | IL-2 | | CD16 | | IL-2 + sAJ2 | |
| | Mean | SD | Mean | SD | Mean | SD | Mean | SD |
| Patient on | 0.0 | 0.0 | 8.9 | 11.3 | 38.5 | 53.6 | 43.9 | 46.8 |
| Cannabis | | | | | | | | |
| Healthy | 0.0 | 0.0 | 26.3 | 56.7 | 28.9 | 57.4 | 78.7 | 111.7 |
| donor | | | | | | | | |
| All cancer | 0.0 | 0.0 | 24.3 | 30.5 | 61.9 | 70.4 | 145.2 | 199.3 |
| patient | | | | | | | | |
| Healthy | 0.0 | 0.0 | 53.8 | 79.8 | 61.4 | 107.4 | 183.0 | 235.8 |
| donor | | | | | | | | |
| | | | | | IL-2 + anti- | | | |
| | untreated | | IL-2 | | CD16 | | IL-2 + sAJ2 | |
| | Mean | SE | Mean | SE | Mean | SE | Mean | SE |
| Patient on | 0.0 | 0.0 | 8.9 | 4.3 | 38.5 | 16.9 | 43.9 | 19.1 |
| Cannabis | | | | | | | | |
| Healthy | 0.0 | 0.0 | 26.3 | 20.0 | 28.9 | 19.1 | 78.7 | 39.5 |
| donor | | | | | | | | |
| All cancer | 0.0 | 0.0 | 24.3 | 11.5 | 61.9 | 23.5 | 145.2 | 81.4 |
| patient | | | | | | | | |
| Healthy | 0.0 | 0.0 | 53.8 | 23.0 | 61.4 | 24.0 | 183.0 | 58.9 |
| donor | | | | | | | | |

### Example 9

### Increased IFN-γ Release from the NK Cells from Patients on Cannabis as Compared to Healthy Controls when Compared to Data from Patients without the Use of Cannabis

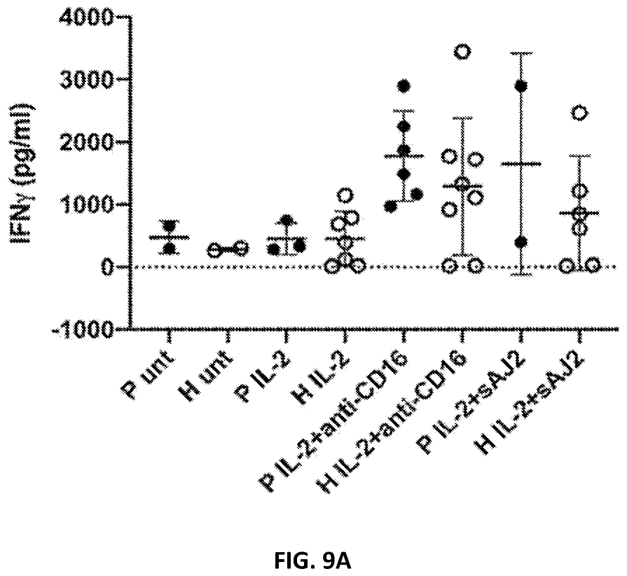

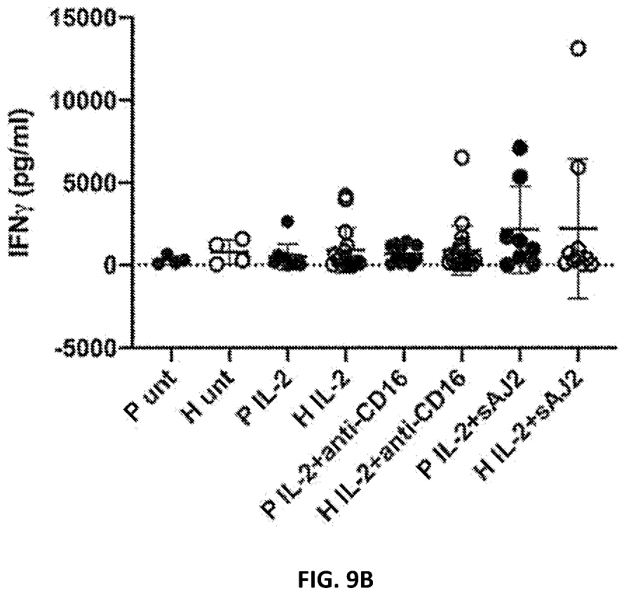

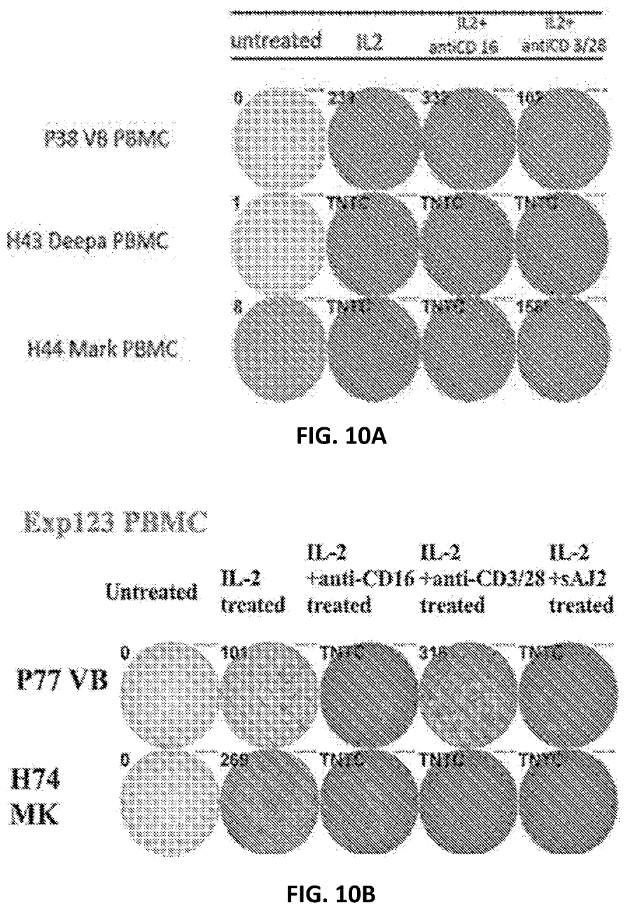

Purified NK cells were isolated as described in the M&M section and ELISA was conducted to determine the amount of IFN-γ secretion in the supernatants. NKs were treated with IL-2 (1000 U/ml) or the combination of IL-2 and anti-CD16mAb (3 μg/ml) or IL-2 in combination with sAJ2. The levels of IFN-γ secretion was variable in patient's NKs on Cannabis when compared to healthy controls. ELISA was performed with purified NK cells of Cannabis Patients (FIG. 9A), All Cancer Patients (FIG. 9B).

NK cells from patients on Cannabis demonstrated increased IFN-γ release when compared to healthy controls and when compared to data from patients who did not ingest cannabis. FIGS. 9A-9B and Table 9.

TABLE 9
ELISA NK (Mean with SD or SEM)

| | | | | | IL-2 + anti- | | | |
|---|---|---|---|---|---|---|---|---|
| | untreated | | IL-2 | | CD16 | | IL-2 + sAJ2 | |
| | Mean | SD | Mean | SD | Mean | SD | Mean | SD |
| Patient on | 470.6 | 257.4 | 452.7 | 253.8 | 1774.0 | 722.9 | 1649.0 | 1769.0 |
| Cannabis | | | | | | | | |
| Healthy | 279.5 | 27.0 | 451.5 | 434.5 | 1289.0 | 1098.0 | 864.2 | 912.0 |
| donor | | | | | | | | |
| All cancer | 160.8 | 117.4 | 165.1 | 122.9 | 620.6 | 388.3 | 227.1 | 214.5 |
| patient | | | | | | | | |
| Healthy | 168.7 | 124.3 | 194.5 | 133.8 | 759.7 | 594 | 214.4 | 242.8 |
| donor | | | | | | | | |

| | | | | | IL-2 + anti- | | | |
|---|---|---|---|---|---|---|---|---|
| | untreated | | IL-2 | | CD16 | | IL-2 + sAJ2 | |
| | Mean | SE | Mean | SE | Mean | SE | Mean | SE |
| Patient on | 470.6 | 182.0 | 8.9 | 4.3 | 38.5 | 16.9 | 43.9 | 19.1 |
| Cannabis | | | | | | | | |
| Healthy | 279.5 | 19.1 | 26.3 | 20.0 | 28.9 | 19.1 | 78.7 | 39.5 |
| donor | | | | | | | | |
| All cancer | 160.8 | 67.78 | 165.1 | 61.45 | 620.6 | 194.1 | 227.1 | 151.7 |
| patient | | | | | | | | |
| Healthy | 168.7 | 71.79 | 194.5 | 59.84 | 759.7 | 265.7 | 214.4 | 140.2 |
| donor | | | | | | | | |

### Example 10

### Cytotoxicity Assay on Patient PMBCs Pre- and Post-Cannabis Administration

Chromium release assays were performed on PMBCs of cancer patients before cannabis administration and after cannabis administration. The PMBCs were either untreated or were treated with one of the following: IL-2; IL-2 and anti-CD16 antibody; or IL-2 and probiotic bacteria (sAJ2). The results are summarized in Table 10 (pre-cannabis administration) and Table 11 (post-cannabis administration).

Briefly, in the chromium release assay, target cells (T) were first incubated with ⁵¹Cr. Then the target cells were incubated with effector cells (T cells; E). If the effector cells kill the targets, then the target cells will release the ⁵¹Cr, which will be detected with a gamma counter.

TABLE 10
Chromium PBMC (pre-Cannabis)

| | | | IL-2 | IL-2 + anti- | IL-2 + sAJ2 |
|---|---|---|---|---|---|
| E:T | | Untreated | treated | CD16 treated | treated |
| | P38* | 23.4 | 54 | 22.9 | 37.1 |
| | H43 | 6.3 | 34.4 | 21.8 | 32.3 |
| | H44 | 10.6 | 18.9 | 29.7 | 12.7 |
| | P38 | 11.5 | 35.8 | 12.2 | 23.4 |
| | H43 | 2.8 | 18.3 | 11.2 | 18.3 |
| | H44 | 4.2 | 2.8 | 17.4 | 4.5 |

*P38 is patient P77 before treatment with cannabis oils.

TABLE 11
Chromium PBMC (post-Cannabis)

| | | | IL-2 | IL-2 + anti- | IL--2 + anti- | IL-2 + sAJ2 |
|---|---|---|---|---|---|---|
| E:T | | untreated | treated | CD16 treated | CD3/28 treated | treated |
| 10:1 | P77 | 7.1 | 44.7 | 36.9 | 48.5 | 53.3 |
| | H74 | 13.3 | 48.6 | 12.6 | 63.4 | 41.4 |
| 5:1 | P77 | 6.0 | 28.3 | 25.6 | 30.8 | 32.1 |
| | H74 | 9.0 | 31.5 | 10.5 | 46.7 | 27.6 |
| 2.5:1 | P77 | 1.3 | 17.7 | 15.2 | 17.4 | 17.6 |
| | H74 | 4.5 | 20.7 | 6.3 | 28.0 | 16.0 |
| 1.25:1 | P77 | 1.1 | 7.7 | 3.2 | 9.4 | 6.3 |
| | H74 | 2.4 | 9.0 | 1.2 | 16.1 | 8.8 |

TABLE 12
ELISA PBMC (pre-Cannabis)

| | | IL-2 | IL-2 + anti- | IL-2 + sAJ2 |
|---|---|---|---|---|
| | Untreated | treated | CD16 treated | treated |
| P38* | 25.7 | 85.1 | 62.5 | 63.3 |
| H43 | 25.0 | 607.9 | 688.0 | 1444.3 |
| H44 | 86.1 | 1742.3 | 2248.4 | 3474.5 |
| Fold increase | — | 7.1 | 11 | 23 |
| healthy | | | | |
| H43/patient | | | | |
| Fold increase | — | 20.5 | 36 | 55 |
| healthy | | | | |
| H44/patient | | | | |

*P38 is patient P77 before treatment with cannabis oils.

TABLE 13
ELISA PBMC (post-Cannabis)

| | | | IL-2 + anti- | IL-2 + anti- | |
|---|---|---|---|---|---|
| | un- | IL-2 | CD16 | CD3/28 | IL-2 + |
| | treated | treated | treated | treated | sAJ2 |
| P77 | 25.9 | 2135.3 | 7289.4 | 2655.3 | 9741.8 |
| H74 | 484.3 | 5381.1 | 6120.8 | 10495.0 | 11422.4 |
| Fold increase | — | 2.5 | ..83 | 3.9 | 1.17 |
| healthy | | | | | |
| H74/patient | | | | | |

### Example 11

### Treatment of Cancer Patients with Cannabis Oils

### Patient 24

Patient 24 is a 20 year-old male that was diagnosed in January 2013 with Stage 4 osteosarcoma. The patient underwent a leg amputation, lung and back surgeries to remove malignant tissues, and removal of vertebra and ribs between 2013 and 2015. This patient suffered from constant back pain and lost movement and usage of his right hand. This patient had not previously been administered cannabis oil. Starting in April 2015, the patient was administered cannabis oils, as straight oils, in the following dosages:
85% CBD Oil: 3× per day (0.25 ml per dose)
50% THC Oil: 3× per day (0.13 ml per dose)
or
CBD 148 MG every 8 hours
THC 65 MG every 8 hours

This patient has less pain, fewer fevers and is cancer free for nearly five years. The patient became cancer free within 120 days.

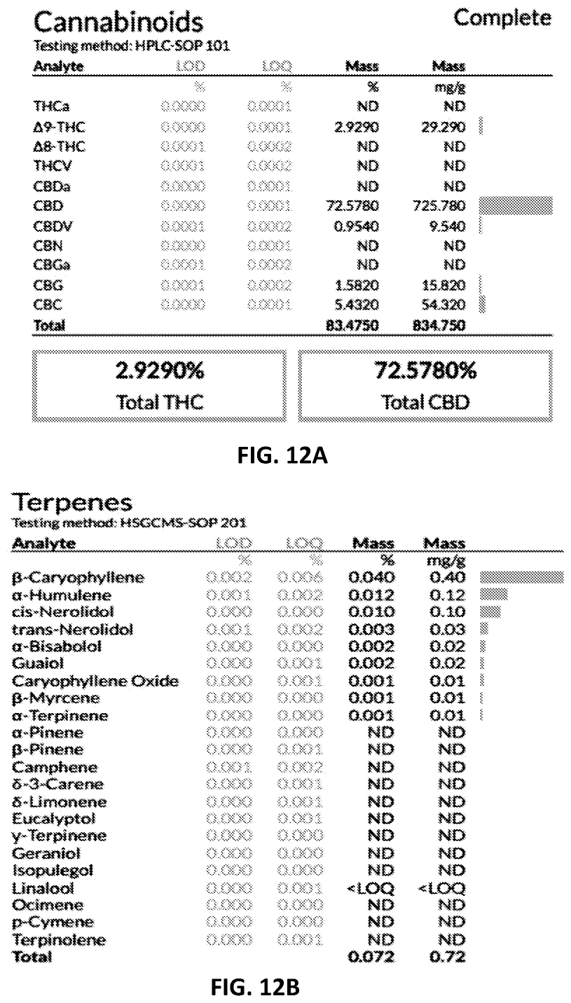

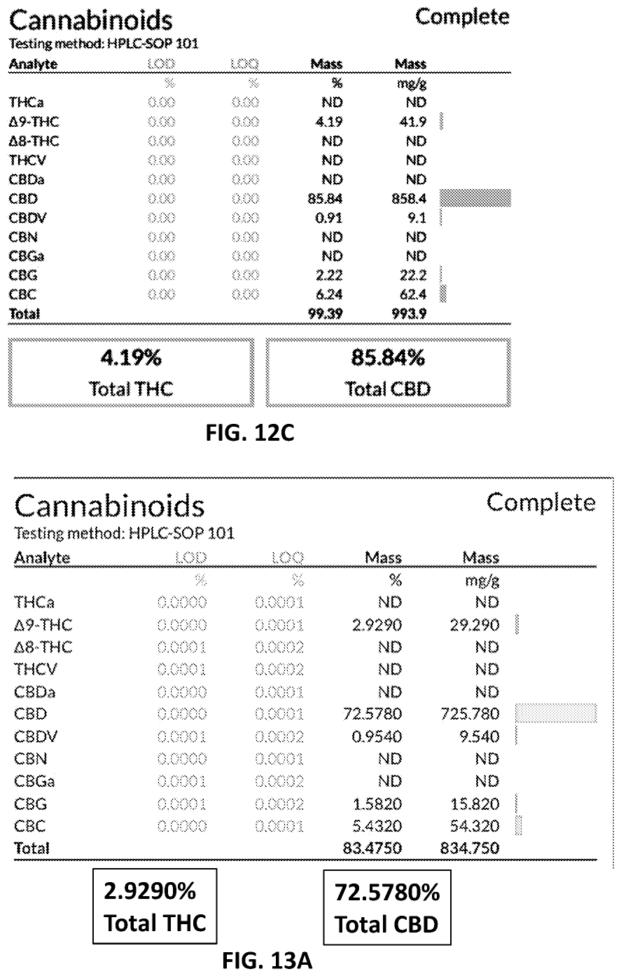

The CBD oil shown in FIG. 12A was administered to the patient.

The administered CBD tincture of cannabis oils comprised the terpene components shown in FIG. 12B.

The THC oils comprised the following cannabinoid components:

TABLE 14
Cannabinoid Profile

| | Wt. % | mg/gram | mg/mL |
|---|---|---|---|
| Δ⁹-THC Max. | 52.20 | 522.04 | NT |
| CBD Max. | 7.67 | 76.71 | NT |
| CBG Max. | 2.51 | 25.13 | NT |
| Δ⁹-THC | 50.21 | 502.07 | NT |
| Δ⁹-THCA | 2.28 | 22.77 | NT |
| CBD | 6.04 | 60.41 | NT |
| CBDA | 1.86 | 18.58 | NT |
| CBG | 2.51 | 25.13 | NT |
| CBGA | ND | ND | NT |
| CBN | 0.91 | 9.12 | NT |
| CBC | 1.48 | 14.80 | NT |
| Δ8-THC | 1.22 | 12.16 | NT |

The administered THC tincture of cannabis oils comprised the following terpene components:

TABLE 15

| Terpenes | mg/g |
|---|---|
| α-Bisabolol | 0.38 |
| β-Caryophyllene | 3.48 |
| Caryophyllene oxide | <LLOQ |
| α-Humulene | 0.89 |
| Limonene | 2.28 |
| Linalool | 0.62 |
| Myrcene | 1.26 |
| α-Pinene | 0.22 |
| β-Pinene | 0.48 |
| Terpinolene | 1.61 |
| Sum of Terpenes | 11.22 |

The CBD oil shown in FIG. 12C was administered to the patient if the above-described CBD oil was not available.

The administered CBD tincture of cannabis oils comprised the following terpene components:

TABLE 16

| Terpenes | mg/g |
|---|---|
| α-Bisabolol | 3.85 |
| β-Caryophyllene | 7.51 |
| Caryophyllene oxide | <LLOQ |
| α-Humulene | 1.60 |
| Limonene | 5.62 |
| Linalool | 0.45 |
| Myrcene | 6.36 |
| α-Pinene | 2.05 |
| β-Pinene | 2.14 |
| Terpinolene | 0.11 |
| Sum of Terpenes | 29.69 |

### Patient 56

Patient P56 is a 65 year old female who was diagnosed in January 2017 with ovarian cancer 3C and underwent a total hysterectomy and apendectomy. The patient has a BRCA2 genetic mutation. The patient is ingesting 600 mg per day of lynparza, an inhibitor of the enzyme poly ADP ribose polymerase (PARP) inhibitor, 30 mg per day of cymbalta and 50 mg per day of trazadone. The patient previously ingested CBD and THC during a first round of chemotherapy in June 2017 for five months and became cancer free. The patient stopped treatment and got off of cannabis, then had a recurrence of cancer in April 2018. Since May 2018 she has administered THC/CBD 20:1 at night only (THC 20 mg/ml 1× per day) and CBD oil 50 mg 3× per day continuously (50 mg/ml CBD and 20 mg/ml THC). The effects of cannabis treatment for this patient included tumor reduction (shown on CT scan), less nausea and better sleep. The patient is now cancer free again, and has remained cancer free while continuing to stay on her cannabis regimen.

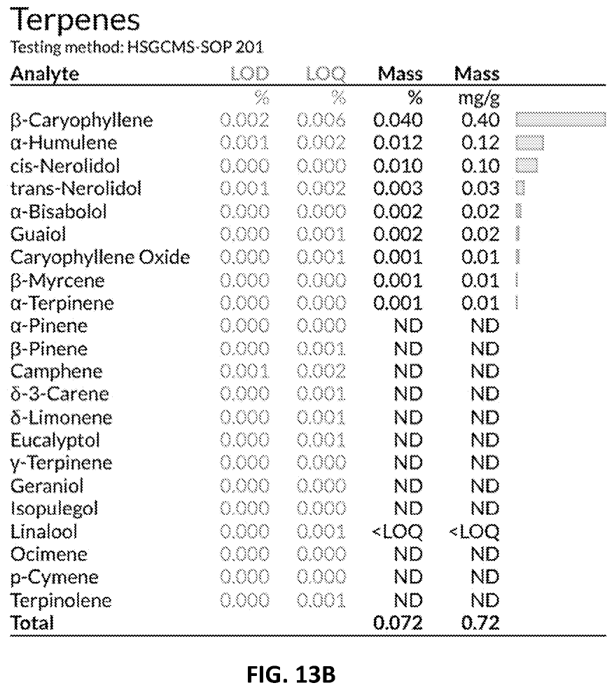

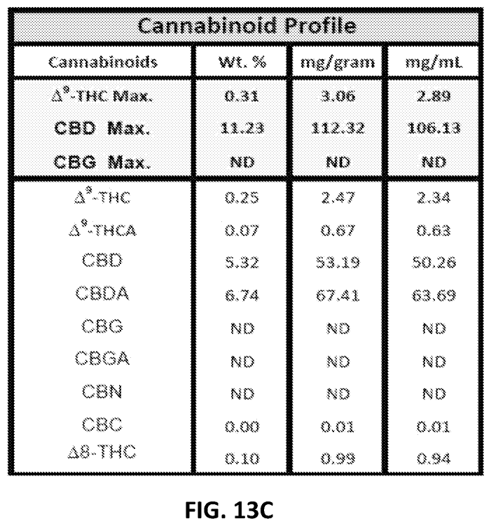

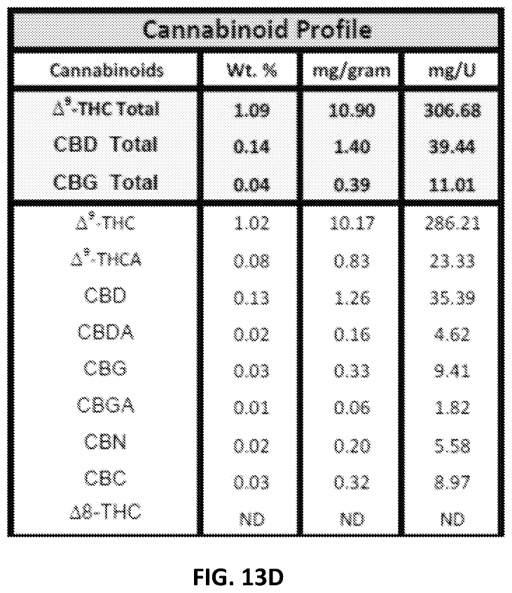

The administered CBD oil comprised the cannabinoid components shown in FIG. 13A.

The administered CBD oil comprised the terpene components shown in FIG. 13B.

The administered 50 mg/ml CBD tincture comprised the following cannabinoid components:

TABLE 17
Cannabinoid Profile

| Cannabinoids | Wt. % | mg/gram | mg/mL |
|---|---|---|---|
| Δ⁹-THC Total | .24 | 2.42 | 2.20 |
| CBD Total | 5.50 | 55.07 | 50.41 |
| CBG Total | .02 | .23 | .21 |
| Δ⁹-THC | 0.12 | 1.22 | 1.11 |
| Δ⁹-THCA | 0.14 | 1.37 | 1.25 |
| CBD | 5.50 | 55.07 | 50.41 |
| CBDA | ND | ND | ND |
| CBG | 0.03 | 0.33 | 0.30 |
| CBGA | ND | ND | ND |
| CBN | ND | ND | ND |
| CBC | 0.15 | 1.50 | 1.36 |
| Δ8-THC | ND | ND | ND |

The administered CBD tincture of cannabis oils comprised the following terpene components:

TABLE 18

| Terpenes | mg/g |
|---|---|
| α-Bisabolol | 3.85 |
| β-Caryophyllene | 7.51 |
| Caryophyllene oxide | <LLOQ |
| α-Humulene | 1.60 |
| Limonene | 5.62 |
| Linalool | 0.45 |
| Myrcene | 6.36 |
| α-Pinene | 2.05 |
| β-Pinene | 2.14 |
| Terpinolene | 0.11 |
| Sum of Terpenes | 29.69 |

CBD Bubblegum flavor (100 MG/ML) comprised the cannabinoid components shown in FIG. 13C. This is a 100 mg/mL tincture that she took in 2017. This was during the patient's first cancer treatment, not the second.

The THC tincture (10 mg/ml) administered comprised the cannabinoids components shown in FIG. 13D.

The administered 10 mg/mL THC tincture of cannabis oils comprised the following terpene components:

TABLE 19

| Terpenes | mg/g |
|---|---|
| α-Bisabolol | 0.51 |
| β-Caryophyllene | 1.36 |
| Caryophyllene oxide | <LLOQ |
| α-Humulene | 0.33 |
| Limonene | 0.89 |
| Linalool | 0.10 |
| Myrcene | 0.92 |
| α-Pinene | 0.29 |
| β-Pinene | 0.31 |
| Terpinolene | 0.08 |
| Sum of Terpenes | 4.80 |

### Patient P23

Patient P23 is a 8 year old female that was diagnosed with an astrocytoma brain stem tumor in February 2014, and was given a terminal diagnosis with a two year life expectancy. The patient has a BRAF genetic mutation. The patient underwent chemotherapy from August 2014 to October 2015 and was in a BRAF trial from January 2017 to October 2018. The patient has been administered Mekinist (0.5 mg pill 1× per day in the morning) from January 2019 to the present. This patient has been administered cannabis oils for five years and currently is administered the following cannabis oils:
CBD 300 mg ix a day,
CBDAA 1× per day 1.0 on syringe full at 309 mg
THC 2× per day 150 mg each dose (total 300 mg, per day),
THA 2× per day 1.0 on syringe full at 750 mg

The patient is noticeably feeling better and has some symptom improvement: sleeps better, has no more pain and is off opioids, is less nausea and demonstrates hunger, and has seen significant tumor reduction on multiple occasions. The patient has remained on cannabis, her tumor remains stable and she is doing great. The administered THC oil comprised the following cannabinoid components:

TABLE 20
Cannabinoid Test Results Nov. 11, 2018
Cannabinoid analysis utilizing High Performance
Liquid Chromatography (HPLC, QSP 5-4-4-4)

| | | | LOD | LOQ |
|---|---|---|---|---|
| | mg/g | % | mg/g | mg/g |
| THC | 778.3 | 77.83 | 0.017 | 0.2 |
| THCa | 39.4 | 3.94 | 0.02 | 0.2 |
| CBD | 1.6 | 0.16 | 0.012 | 0.2 |
| CBDa | <LOQ | <LOQ | 0.012 | 0.2 |
| CBN | 9.8 | 0.98 | 0.006 | 0.2 |
| CBDV | ND | ND | 0.0034 | 0.2 |
| CBDVa | ND | ND | 0.014 | 0.2 |
| CBG | 12.4 | 1.24 | 0.012 | 0.2 |
| CBGa | 0.3 | 0.03 | 0.017 | 0.2 |
| THCV | 3.3 | 0.33 | 0.009 | 0.2 |
| Δ8 - THC | ND | ND | 0.021 | 0.2 |
| CBC | 8.5 | 0.85 | 0.011 | 0.2 |
| Sum of Cannabinoids: | 853.6 | 85.36 | | |
| Total THC (Δ9THC + 0.877*THCa) | 812.9 | 81.29 | | |
| Total CBD (CBD + 0.877*CBDa) | 1.6 | 0.16 | | |

The administered THC tincture of cannabis oils comprised the following terpene components:

TABLE 21

| Terpenes | mg/g |
|---|---|
| α-Bisabolol | 0.36 |
| β-Caryophyllene | 3.46 |
| Caryophyllene oxide | <LLOQ |
| α-Humulene | 0.89 |
| Limonene | 2.26 |
| Linalool | 0.62 |
| Myrcene | 1.26 |
| α-Pinene | 0.22 |
| β-Pinene | 0.48 |
| Terpinolene | 1.61 |
| Sum of Terpenes | 11.22 |

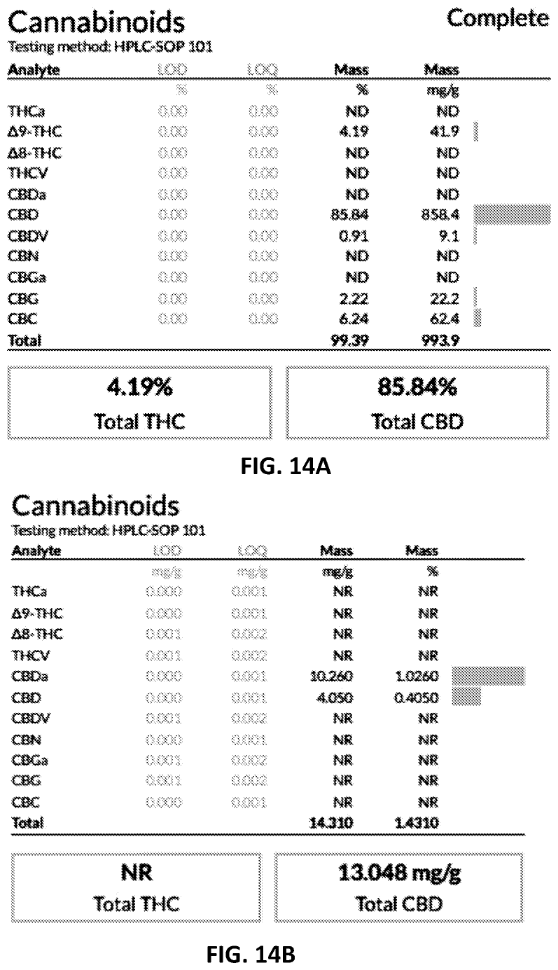

The CBD oil comprised the cannabinoid components shown in FIG. 14A.

The administered CBD tincture of cannabis oils comprised the following terpene components:

TABLE 22

| Terpenes | mg/g |
|---|---|
| α-Bisabolol | 3.85 |
| β-Caryophyllene | 7.51 |
| Caryophyllene oxide | <LLOQ |
| α-Humulene | 1.60 |
| Limonene | 5.62 |
| Linalool | 0.45 |
| Myrcene | 6.36 |
| α-Pinene | 2.05 |
| β-Pinene | 2.14 |
| Terpinolene | 0.11 |
| Sum of Terpenes | 29.69 |

The CBDa oil comprised the cannabinoid components shown in FIG. 14B.

The THCa tincture comprised the following cannabinoid components:

TABLE 23
Cannabinoid Profile

| Cannabinoids | Wt. % | mg/gram | mg/U |
|---|---|---|---|
| Δ⁹-THC Total | 2.77 | 27.71 | 756.19 |
| CBD Total | 0.05 | 0.54 | 14.80 |
| CBG Total | 0.14 | 1.42 | 38.86 |
| Δ9-THC | 0.38 | 3.76 | 102.47 |
| Δ9-THCA | 2.73 | 27.31 | 745.19 |
| CBD | 0.05 | 0.52 | 14.08 |
| CBDA | 0.00 | 0.03 | 0.82 |
| CBG | 0.02 | 0.16 | 4.49 |
| CBGA | 0.14 | 1.43 | 39.15 |
| CBN | ND | ND | ND |
| CBC | 0.01 | 0.07 | 2.03 |
| Δ8-THC | ND | ND | ND |

The THCa tincture comprised the following terpene components:

TABLE 24

| | | | LOD | LOQ |
|---|---|---|---|---|
| | mg/g | % | mg/g | mg/g |
| [symbol] Bisabolol | 11.48 | 1.148 | 0.21 | 0.67 |
| [symbol] Pinene | 242.09 | 24.209 | 0.21 | 0.67 |
| 3 Carene | 1.29 | 0.129 | 0.21 | 0.67 |
| Borneol | 1.01 | 0.101 | 0.21 | 0.67 |
| [symbol] Caryophyllene | 91.02 | 9.102 | 0.21 | 0.67 |
| Geraniol | ND | ND | 0.21 | 0.67 |
| [symbol] Humulene | 42.89 | 4.289 | 0.21 | 0.67 |
| Terpinolene | ND | ND | 0.21 | 0.67 |
| Valencene | <LOQ | <LOQ | 0.21 | 0.67 |
| Menthol | ND | ND | 0.21 | 0.67 |
| Nerolidol | 2.43 | 0.243 | 0.21 | 0.67 |
| Camphene | 4.13 | 0.413 | 0.21 | 0.67 |
| Eucalyptol | ND | ND | 0.21 | 0.67 |
| [symbol] Cedrene | ND | ND | 0.21 | 0.67 |
| Camphor | ND | ND | 0.21 | 0.67 |
| (−)-Isopulegol | ND | ND | 0.21 | 0.67 |
| Sabinene | ND | ND | 0.21 | 0.67 |
| [symbol] Terpinene | ND | ND | 0.21 | 0.67 |
| [symbol] Terpinene | ND | ND | 0.21 | 0.67 |
| Linalool | 9.86 | 0.986 | 0.21 | 0.67 |
| Limonene | 80.60 | 8.060 | 0.21 | 0.67 |
| Myrcene | 273.18 | 27.318 | 0.21 | 0.67 |
| Fenchol | 2.28 | 0.228 | 0.21 | 0.67 |
| [symbol] Phellandrene | 12.13 | 1.213 | 0.21 | 0.67 |
| Caryophyllene Oxide | 2.25 | 0.225 | 0.21 | 0.67 |
| Terpineol | 2.68 | 0.268 | 0.21 | 0.67 |
| [symbol] Pinene | 104.56 | 10.456 | 0.21 | 0.67 |
| R-(+)-Pulegone | 0.83 | 0.083 | 0.21 | 0.67 |
| Geranyl Acetate | 2.31 | 0.231 | 0.21 | 0.67 |
| Citronellol | 1.04 | 0.104 | 0.21 | 0.67 |
| p-Cymene | ND | ND | 0.21 | 0.67 |
| Ocimene | ND | ND | 0.21 | 0.67 |
| Guaiol | ND | ND | 0.21 | 0.67 |
| Phytol | 9.60 | 0.960 | 0.21 | 0.67 |
| Isoborneol | ND | ND | 0.21 | 0.67 |
| Total Terpene Concentration: | 897.66 | 89.766 | | |

### Patient P57

Patient P57 was a 13 year old male that was diagnosed with osteosarcoma in February 2016. The patient underwent traditional MAP chemotherapy (Cisplatin, HD methotrexate and doxorubicin) from February 2016 to October 2016, immunotherapy with Mifamurtide (MPT) from August 2016 to August 2017, continuous Ifosomide from April 2017 to June 2017, and from June 2017 to the present is administered Gemcitabine (chemo), Abraxane (chemo), Dimosumab (a PDL-1 inhibitor) and Nivolumab (immunotherapy).

For three months in 2017, the patient was administered:
Month 1—CBD 150 mg; CBDA 20 mg
Month 2—CBD 150 mg; CBDA 20 mg, THCa 25 mg and THC 100 mg
Month 3—CBD 150 mg; CBDA 20 mg and THCa 25 mg.
   The patient will be administered cannabis oils as follows:
CBD (100 mg/1 ml):
Week 1: 20 mg every 8 hours
Week 2: 40 mg every 8 hours
Week 3: 80 mg every 8 hours
Week 4: 165 mg every 8 hours
The CBD administration goal is 500 mg per day.
CBDa (30 mg/ml)
Week 1: 15 mg every 8 hours
Week 2: 30 mg every 8 hours
The CBDA administration goal is 90 mg per day.
THCA (20 mg/1 ml)
Week 1: 10 mg every 8 hours
Week 2: 20 mg every 8 hours
The THCA administration goal is 60 mg per day.
THC (25 mg/ml) to start
Week 1: 5 mg every 8 hours
Week 2: 10 mg every 8 hours
Week 3: 15 mg every 8 hours
Week 4: 20 mg every 8 hours
Week 5: 25 mg every 8 hours
Week 6: 30 mg every 8 hours
Week 7: 40 mg every 8 hours
Week 8: 50 mg every 8 hours
This patient died before the proposed treatment.

### Patient P82

Patient P82 is a 30 year old male that was diagnosed with non-Hodgkins Large B-cell lymphoma in June 2016. The patient underwent two rounds of chemotherapy, stem cell transplant, localized brain radiation and brentuximab immunotherapy.

The patient has been administered hemp CBD oil 50 mg/ml (2×5 ml per day) for eight months. The patient is noticeably feeling better with some symptom improvement and better sleep. He is unable to take cannabis based oils due to sensitivity to the psychoactivity. The patient stopped taking the hemp oil when his cancer returned, completed another round of immunotherapy which did not work. He is now evaluating additional treatment options.

The administered Hemp CBD oil (tincture) (50 mg) comprised the following cannabinoid components:

TABLE 25
Cannabinoid Profile

| Cannabinoids | Wt. % | mg/gram | mg/mL |
|---|---|---|---|
| Δ⁹-THC Total | .24 | 2.42 | 2.20 |
| CBD Total | 5.50 | 55.07 | 50.41 |
| CBG Total | .02 | .23 | .21 |
| Δ⁹-THC | 0.12 | 1.22 | 1.11 |
| Δ⁹-THCA | 0.14 | 1.37 | 1.25 |
| CBD | 5.50 | 55.07 | 50.41 |
| CBDA | ND | ND | ND |
| CBG | 0.03 | 0.33 | 0.30 |
| CBGA | ND | ND | ND |
| CBN | ND | ND | ND |
| CBC | 0.15 | 1.50 | 1.36 |
| Δ8-THC | ND | ND | ND |

The administered CBD tincture of cannabis oils comprised the following terpene components:

TABLE 26

| Terpenes | mg/g |
|---|---|
| α-Bisabolol | 3.85 |
| β-Caryophyllene | 7.51 |
| Caryophyllene oxide | <LLOQ |
| α-Humulene | 1.60 |
| Limonene | 5.62 |
| Linalool | 0.45 |
| Myrcene | 6.36 |
| α-Pinene | 2.05 |
| β-Pinene | 2.14 |
| Terpinolene | 0.11 |
| Sum of Terpenes | 29.69 |

### Patient 22

Patient P22 is a 7 year old female that was diagnosed with an optic pathway glioma in 2013 at 8½ months old. She was given a 90% survival rate, but this type of tumor has an 85% recurrence rate. She began taking low doses THC and CBD at 9 months old totaling 26mgs THC+13mgs CBD. After the tumor hit a growth spurt during a watch and wait period Carboplatin and Vincristine chemotherapy was administered and the cannabis was increased to 400mgs at a 2:1 ratio. (266mgs of THC+133mgs of CBD. 6 months later the ratio became a 1:1 at 200mgs of CBD and THC. After 13 months of chemo she saw close to 90% overall reduction in the tumor, vision was preserved after being given 100% chance of going blind, and she stopped needing blood transfusions 3 months before ending treatment after she had previously needed them every cycle for 4 months. The doctors took her off of chemotherapy despite the tumor still shrinking on the last scan and 8 months later the tumor grew back.

The patient has since used Avastin+Vincristine/Carboplatin, Vinblastine, Mek 162 inhibitor, Trametinib, and TPCV. She has experienced incredible immunological responses in how her counts recover faster than what is considered normal, she has kept on weight and her hair has never fallen out, she was admitted twice to the hospital for pneumonia which was determined via X-rays, but was released from the hospital the next afternoon with no antibiotics prescribed and a full recovery. She has experienced reductions in nausea, appetite stimulation, and pain reduction. Patient P22 had a 7 hour brain surgery on Apr. 23, 2018 during which a golf ball sized tumor was removed and an incision was made from the middle of her forehead to the bottom of her right ear. She experienced no visible swelling or bruising anywhere on her face or eyes, saw an extreme reduction in pain after being given cannabis post-op, was released from the hospital in under 48 hours and was told she could go back to school in 1-2 days instead of 1-2 weeks.

Over the 6 years of treatment the protocol has been adjusted in an effort to find the best protocol. The patient's tumors are currently stable and the patient is about to complete the TPCV protocol. Her seizure activity is stable using cannabis and Keppra when she otherwise began having cluster seizures when cannabis was removed. She is growing normally, has had no neurological deficits except for an issue with fine motor skills due to tumor damage, has a very healthy appetite, a full head of hair and has not been emotionally affected in ways that would otherwise be expected by over 6 years of treatment. She has lost much of her vision which is believed to be due to a brain tumor surgery that resulted in a large post-surgical cyst pressing against her nerves that had to be surgically drained. She had otherwise retained much of her vision prior.

The patient's administration dosing schedule from April 2018 to April 2019 was as follows:

TABLE 27

| | Administration |
|---|---|
| Cannabinoid Administered | Date |
| THCa = 20 mg 2x daily = 40 mg daily | Apr. 10, 2019 |
| CBDa = 10 mg 2x daily = 20 mg daily | Apr. 10, 2019 |
| THC = 80 mg + 100 mg + 120 mg = 300 mg daily | Apr. 10, 2019 |
| CBD = 126 mg 3x daily = 378 mg daily | Apr. 10, 2019 |
| THCa = 20 mg 2x daily = 40 mg daily | Oct. 15, 2018 |
| CBDa = 10 mg 2x daily = 20 mg daily | Oct. 15, 2018 |
| THC = 80 mg + 100 mg + 120 mg = 300 mg daily | Oct. 15, 2018 |
| CBD = 165 mg 3x daily = 495 mg daily, | Oct. 15, 2018 |
| THCa = 30 mg 2x daily = 60 mg daily | Apr. 15, 2018 |
| CBDa = 10 mg 2x daily = 20 mg daily | Apr. 15, 2018 |
| THC = 60 mg + 80 mg + 100 mg = 240 mg daily | Apr. 15, 2018 |
| CBD = 165 mg 3x daily = 495 mg daily | Apr. 15, 2018 |

The CBD oil that was administered starting Apr. 10, 2019 comprised the following cannabinoid components:

TABLE 28
Cannabinoid Profile

| Cannabinoids | Wt. % | mg/gram | mg/mL |
|---|---|---|---|
| Δ⁹-THC Total | 0.33 | 3.26 | 3.08 |
| CBD Total | 10.37 | 103.72 | 98.02 |
| CBG Total | 0.17 | 1.66 | 1.57 |
| Δ⁹-THC | 0.33 | 3.26 | 3.08 |
| Δ⁹-THCA | ND | ND | ND |
| CBD | 10.37 | 103.72 | 98.02 |
| CBDA | ND | ND | ND |
| CBG | 0.17 | 1.66 | 1.57 |
| CBGA | ND | ND | ND |
| CBN | 0.02 | 0.17 | 0.16 |
| CBC | 0.74 | 7.41 | 7.00 |
| Δ8-THC | ND | ND | ND |

The administered CBD tincture of cannabis oils comprised the following terpene components:

TABLE 29

| Terpenes | mg/g |
|---|---|
| α-Bisabolol | 3.85 |
| β-Caryophyllene | 7.51 |
| Caryophyllene oxide | <LLOQ |
| α-Humulene | 1.60 |
| Limonene | 5.62 |
| Linalool | 0.45 |
| Myrcene | 6.36 |
| α-Pinene | 2.05 |
| β-Pinene | 2.14 |
| Terpinolene | 0.11 |
| Sum of Terpenes | 29.69 |

The CBDA oil (22 mg/ml) that was administered starting Apr. 10, 2019 (this CBDa is representative of other CBDA oils administered) comprised the following cannabinoid components:

TABLE 30
Cannabinoid Profile

| Cannabinoids | Wt. % | mg/gram | mg/mL |
|---|---|---|---|
| Δ⁹-THC Max. | 0.10 | 1.02 | 0.93 |
| CBD Max. | 2.24 | 22.37 | 20.36 |
| CBG Max. | 0.02 | 0.24 | 0.22 |
| Δ⁹-THC | 0.03 | 0.32 | 0.29 |
| Δ⁹-THCA | 0.08 | 0.79 | 0.72 |
| CBD | 0.30 | 3.01 | 2.74 |
| CBDA | 2.21 | 22.07 | 20.09 |
| CBG | ND | ND | ND |
| CBGA | 0.03 | 0.28 | 0.25 |
| CBN | ND | ND | ND |
| CBC | ND | ND | 0.01 |
| Δ8-THC | 0.02 | 0.16 | 0.15 |

The THC tincture (50 mg/ml) that was administered starting Apr. 10, 2019 comprised the following cannabinoid components:

TABLE 31
Cannabinoid Profile

| Cannabinoids | Wt. % | mg/gram | mg/mL |
|---|---|---|---|
| Δ⁹-THC Total | 5.61 | 56.15 | 52.89 |
| CBD Total | 0.06 | 0.60 | 0.57 |
| CBG Total | 0.08 | 0.84 | 0.79 |
| Δ⁹-THC | 5.48 | 54.85 | 51.67 |
| Δ⁹-THCA | 0.15 | 1.48 | 1.40 |
| CBD | 0.06 | 0.60 | 0.57 |
| CBDA | ND | ND | ND |
| CBG | 0.08 | 0.84 | 0.79 |
| CBGA | ND | ND | ND |
| CBN | 0.06 | 0.61 | 0.58 |
| CBC | 0.07 | 0.69 | 0.65 |
| Δ8-THC | ND | ND | ND |

The administered THC tincture of cannabis oils comprised the following terpene components:

TABLE 32

| Terpenes | mg/g |
|---|---|
| α-Bisabolol | 0.38 |
| β-Caryophyllene | 3.48 |
| Caryophyllene oxide | <LLOQ |
| α-Humulene | 0.89 |
| Limonene | 2.28 |
| Linalool | 0.62 |
| Myrcene | 1.26 |
| α-Pinene | 0.22 |
| β-Pinene | 0.48 |
| Terpinolene | 1.61 |
| Sum of Terpenes | 11.22 |

The THCa tincture (27 mg/ml) was administered starting Apr. 10, 2019 (this THCa is representative of other THCa tinctures administered to this patient) comprised the following cannabinoids:

The administered THCA oil comprised the following cannabinoid components:

TABLE 33
Cannabinoid Profile

| Cannabinoids | Wt. % | mg/gram | mg/U |
|---|---|---|---|
| Δ⁹-THC Total | 2.77 | 27.71 | 756.19 |
| CBD Total | 0.05 | 0.54 | 14.80 |
| CBG Total | 0.14 | 1.42 | 38.86 |
| Δ9-THC | 0.38 | 3.76 | 102.47 |
| Δ9-THCA | 2.73 | 27.31 | 745.19 |
| CBD | 0.05 | 0.52 | 14.08 |
| CBDA | 0.00 | 0.03 | 0.82 |
| CBG | 0.02 | 0.16 | 4.49 |
| CBGA | 0.14 | 1.43 | 39.15 |
| CBN | ND | ND | ND |
| CBC | 0.01 | 0.07 | 2.03 |
| Δ8-THC | ND | ND | ND |

The administered THCa tincture of cannabis oils comprised the following terpene components:

TABLE 34

| | | | LOD | LOQ |
|---|---|---|---|---|
| | mg/g | % | mg/g | mg/g |
| [symbol] Bisabolol | 11.48 | 1.148 | 0.21 | 0.67 |
| [symbol] Pinene | 242.09 | 24.209 | 0.21 | 0.67 |
| 3 Carene | 1.29 | 0.129 | 0.21 | 0.67 |
| Borneol | 1.01 | 0.101 | 0.21 | 0.67 |
| [symbol] Caryophyllene | 91.02 | 9.102 | 0.21 | 0.67 |
| Geraniol | ND | ND | 0.21 | 0.67 |
| [symbol] Humulene | 42.89 | 4.289 | 0.21 | 0.67 |
| Terpinolene | ND | ND | 0.21 | 0.67 |
| Valencene | <LOQ | <LOQ | 0.21 | 0.67 |
| Menthol | ND | ND | 0.21 | 0.67 |
| Nerolidol | 2.43 | 0.243 | 0.21 | 0.67 |
| Camphene | 4.13 | 0.413 | 0.21 | 0.67 |
| Eucalyptol | ND | ND | 0.21 | 0.67 |
| [symbol] Cedrene | ND | ND | 0.21 | 0.67 |
| Camphor | ND | ND | 0.21 | 0.67 |
| (−)-Isopulegol | ND | ND | 0.21 | 0.67 |
| Sabinene | ND | ND | 0.21 | 0.67 |
| [symbol] Terpinene | ND | ND | 0.21 | 0.67 |
| [symbol] Terpinene | ND | ND | 0.21 | 0.67 |
| Linalool | 9.86 | 0.986 | 0.21 | 0.67 |
| Limonene | 80.60 | 8.060 | 0.21 | 0.67 |
| Myrcene | 273.18 | 27.318 | 0.21 | 0.67 |
| Fenchol | 2.28 | 0.228 | 0.21 | 0.67 |
| [symbol] Phellandrene | 12.13 | 1.213 | 0.21 | 0.67 |
| Caryophyllene Oxide | 2.25 | 0.225 | 0.21 | 0.67 |
| Terpineol | 2.68 | 0.268 | 0.21 | 0.67 |
| [symbol] Pinene | 104.56 | 10.456 | 0.21 | 0.67 |
| R-(+)-Pulegone | 0.83 | 0.083 | 0.21 | 0.67 |
| Geranyl Acetate | 2.31 | 0.231 | 0.21 | 0.67 |
| Citronellol | 1.04 | 0.104 | 0.21 | 0.67 |
| p-Cymene | ND | ND | 0.21 | 0.67 |
| Ocimene | ND | ND | 0.21 | 0.67 |
| Guaiol | ND | ND | 0.21 | 0.67 |
| Phytol | 9.60 | 0.960 | 0.21 | 0.67 |
| Isoborneol | ND | ND | 0.21 | 0.67 |
| Total Terpene Concentration: | 897.66 | 89.766 | | |

### Patient P38/P77

Patient P38 also is called patient P77. P38 is the designation for this patient prior to cannabis oils treatment; P77 is the designation for this patient post-treatment with cannabis oils. Patient P38 is a 47 year old female that was diagnosed with breast cancer Stage II in February 2014 and Stage IV breast cancer with lung metastases in 2018. The patient underwent genetic testing in 2014, but no genetic mutations were found. The patient underwent a successful lumpectomy in March 2014. The patient began therapy with tamoxifen beginning in October 2017. The patient began administration of cannabis oils in September 2018 and is currently administered hemp CBD oil (50 mg/ml) at doses of 100-200 mg CBD 3× per day. The target dose is 300-600 mg/day.

The patient is noticeably feeling better with some symptom improvement and better sleep. The patient's immune system functioning has improved to an extent that is comparable to (or better than) a person who does not have cancer, i.e., a healthy control. (Example 10, Tables 10-13).

The patient's cancer is currently stable after a scan in April 2019, she has much more energy, her appetite has been strong and she has both maintained and gained weight. The administered CBD (50 mg/ml) tincture comprised the following cannabinoid components:

TABLE 35
Cannabinoid Profile

| Cannabinoids | Wt. % | mg/gram | mg/mL |
|---|---|---|---|
| Δ⁹-THC Total | .24 | 2.42 | 2.20 |
| CBD Total | 5.50 | 55.07 | 50.41 |
| CBG Total | .02 | .23 | .21 |
| Δ⁹-THC | 0.12 | 1.22 | 1.11 |
| Δ⁹-THCA | 0.14 | 1.37 | 1.25 |
| CBD | 5.50 | 55.07 | 50.41 |
| CBDA | ND | ND | ND |
| CBG | 0.03 | 0.33 | 0.30 |
| CBGA | ND | ND | ND |
| CBN | ND | ND | ND |
| CBC | 0.15 | 1.50 | 1.36 |
| Δ8-THC | ND | ND | ND |

The administered CBD tincture of cannabis oils comprised the following terpene components:

TABLE 36

| Terpenes | mg/g |
|---|---|
| α-Bisabolol | 0.01 |
| β-Caryophyllene | 3.77 |
| Caryophyllene oxide | 0.01 |
| α-Humulene | 0.89 |
| Limonene | 13.91 |
| Linalool | 0.36 |
| Myrcene | 3.94 |
| α-Pinene | 1.55 |
| β-Pinene | 2.29 |
| Terpinolene | 0.09 |
| Sum of Terpenes | 26.80 |

### Example 12

### Function of NK Cells were Increased in PBMCs and CD3+ T Cells Isolated from Splenocytes of WIN 55,212-2 Treated Hu-BLT Mouse

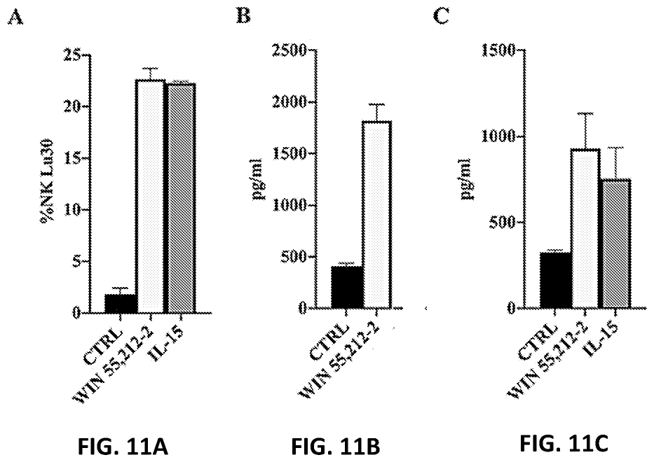

PBMCs (FIG. 11A and FIG. 11B) and CD3+ T cells (FIG. 11C) were isolated from hu-BLT mice after treatment of WIN 55,212-2 (intraperitoneal (IP) injection every 2 days at 2 mg/Kg in DMSO+saline for 27 days before sacrifice) or IL-15 (IP injection of 5 μg per mice for 27 days). PBMCs were isolated from peripheral blood and treated with IL-2 (1000 u/ml) overnight before they were used in 51 Cr release assay. The Lytic units (LU) 30/10⁶ cells were determined by plotting the % cytotoxicity obtained at different effector to target ratios, and using that curve to determine the inverse number of NK cells required to lyse 30% of OSCSCs×100. The numbers of LU were then adjusted based on the % of NK cells obtained by flow cytometric analysis (FIG. 11A). PBMCs and CD3+ T cells culture supernatants were collected and IFN-γ secretions were determined using ELISA (FIG. 11B and FIG. 11C). CD3 T cells were isolated from splenocytes by negative selection of CD3+ T cells using an isolation kit (Stem cell technologies). WIN 55,212-2 treated mice showed higher NK cell mediated cytotoxicity and IFN-γ secretion in both PBMC and CD3+ T cells. IL-15 treated mouse showed higher NK cell mediated cytotoxicity in PBMC and IFN-γ secretion in CD3+ T cells.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for (a) increasing cytotoxicity of a natural killer (NK) cell, (b) preventing or reversing inactivation of cytotoxicity of a natural killer (NK) cell (e.g., an exhausted NK cell), or (c) increasing secretion of interferon gamma (IFN-γ) by a peripheral blood mononuclear cell (PBMC), the method comprising administering to a subject a composition comprising cannabis.

2. The method of claim 1, further comprising administering to the subject:
(a) a composition comprising at least one of anti-CD3 antibodies, anti-CD28 antibodies, IL-2, IL-2 and anti-CD16 antibodies, and IL-2 and sAJ2 bacteria, or a combination thereof, optionally wherein the antibodies are monoclonal antibodies; and/or
(b) at least one cancer therapy.

3. The method of claim 1, wherein the composition comprising cannabis is administered by inhalation, oral administration, sublingual administration, and/or topical administration.

4. The method of claim 3, wherein
(i) the oral administration of the composition comprising cannabis comprises ingesting
(a) a cannabis oil;
(b) a cannabis tincture; and/or
(c) a food and/or beverage, wherein the food and the beverage each comprises the composition comprising cannabis; or
(ii) the inhalation is by smoking or vaporizing.

5. (canceled)

6. The method of claim 2, wherein the composition comprising at least one of anti-CD3 antibodies, anti-CD28 antibodies, IL-2, and IL-2 and anti-CD16 antibodies is administered intravenously.

7. The method of claim 2, wherein the composition comprising cannabis and the composition comprising at least one of anti-CD3 antibodies, anti-CD28 antibodies, IL-2, IL-2 and anti-CD16 antibodies, and IL-2 and sAJ2 bacteria are administered concurrently or sequentially.

8. The method of claim 1, wherein the cannabis comprises CBD, CBDa, THC and/or THCa.

9. The method of claim 1, wherein
(a) the method comprises administering to the subject at least about 0.001 mg/kg and no more than about 100 mg/kg of cannabis;
(b) the subject is administered cannabis at least once a week or at least once a day; and/or
(c) the subject has cancer and/or a tumor.

10-12. (canceled)

13. The method of claim 1, wherein the PBMC is a natural killer (NK) cell, a natural killer T (NKT), a CD4 T helper (TH) lymphocyte, and/or a CD8 cytotoxic cell, optionally wherein the PBMC is a natural killer cell.

14. (canceled)

15. The method of claim 1, wherein the composition comprising cannabis is administered in an amount effective to reactivate an exhausted NK cell.

16-25. (canceled)

26. The method of claim 1, wherein the subject demonstrates decreased secretion of IFN-γ prior to the administration of the composition comprising cannabis.

27-37. (canceled)

38. A method for treating a subject having cancer and/or a tumor, the method comprising:
(a) obtaining a blood or tissue sample of the subject;
(b) determining a level of activation of an NK cell and/or a level of secretion of IFN-γ by a NK cell in the blood or tissue sample of the subject;
(c) comparing the determined level of activation of the NK cell and/or the determined level of secretion of IFN-γ by the NK cell to a control, optionally wherein the control is a level of activation of an NK cell from a healthy subject and/or a level of secretion of IFN-γ by a NK cell in the blood or tissue sample of a healthy subject; and
(d) if the determined level of activation of the NK cell and/or the determined level of secretion of IFN-γ by the NK cell in the blood or tissue sample of the subject is decreased compared to the control, administering to the subject a composition comprising cannabis.

39. The method of claim 38, further comprising administering to the subject:
(a) a composition comprising at least one of anti-CD3 antibodies, anti-CD28 antibodies, IL-2, IL-2 and anti-CD16 antibodies, and IL-2 and sAJ2 bacteria, or a combination thereof, optionally wherein the antibodies are monoclonal antibodies; and/or
(b) at least one cancer therapy.

40. The method of claim 38, wherein the composition comprising cannabis is administered by inhalation, oral administration, sublingual administration, and/or topical administration.

41. The method of claim 40, wherein
(i) the oral administration of the composition comprising cannabis comprises ingesting
(a) a cannabis oil;
(b) a cannabis tincture; and/or
(c) a food and/or beverage, wherein the food and the beverage each comprises the composition comprising cannabis; or
(ii) the inhalation is by smoking or vaporizing.

42. (canceled)

43. The method of claim 39, wherein the composition comprising at least one of anti-CD3 antibodies, anti-CD28 antibodies, IL-2, and IL-2 and anti-CD16 antibodies is administered intravenously.

44. The method of claim 39, wherein the composition comprising cannabis and the composition comprising at least one of anti-CD3 antibodies, anti-CD28 antibodies, IL-2, IL-2 and anti-CD16 antibodies, and IL-2 and sAJ2 bacteria are administered concurrently or sequentially.

45. The method of claim 38, wherein the cannabis comprises CBD, CBDa, THC and/or THCa.

46. The method of claim 38, wherein
(a) the method comprises administering to the subject at least about 0.001 mg/kg and no more than about 100 mg/kg of cannabis; and/or
(b) the subject is administered cannabis at least once a week or at least once a day.

47. (canceled)

48. A method of increasing cytotoxicity of an NK cell or a method of increasing secretion of IFN-γ by the NK cell, the method comprising contacting the NK cell with cannabis, wherein the NK cell is contacted with cannabis in vitro or ex vivo.

49. (canceled)
